(19) United States
(12) Patent Application Publication
(10) Pub. No.: US 2013/0252847 A1
(43) Pub. Date: Sep. 26, 2013

(54) ASSAY ASSEMBLY AND METHOD

(71) Applicants: Ekaterina Olegovna McKenna, Glasgow (GB); Yifan Li, Edinburgh (GB); Andrew Robert Pitt, Glasgow (GB); Anthony John Walton, Edinburgh (GB); William Parkes, Glasgow (GB)

(72) Inventors: Ekaterina Olegovna McKenna, Glasgow (GB); Yifan Li, Edinburgh (GB); Andrew Robert Pitt, Glasgow (GB); Anthony John Walton, Edinburgh (GB); William Parkes, Glasgow (GB)

(21) Appl. No.: 13/991,333

(22) PCT Filed: Dec. 2, 2011
(86) PCT No.: PCT/GB2011/001676

§ 371 Date: Jun. 3, 2013

(30) Foreign Application Priority Data
Dec. 3, 2010 (GB) 1020574.8

(51) Int. Cl. G01N 33/53

(52) U.S. Cl. CPC G01N 33/5306
USPC 506/9; 506/13; 506/43

(57) ABSTRACT

The present invention relates to assay assemblies, components of assay assemblies, methods of determining one or more properties of a sample liquid, and methods of making components of assay assemblies. The present invention allows the properties of very small volumes of sample liquid to be probed, by providing a first surface having at least one sample liquid wettable surface region defined by a sample liquid repelling boundary, and a second surface, opposed to the first surface, having analyte binding agent immobilised thereon.

### FIELD OF THE INVENTION

The present invention relates to assay assemblies, to kits comprising components of assay assemblies, to methods of determining one or more properties of a sample liquid, and to methods of making components of assay assemblies.

### BACKGROUND OF THE INVENTION

Assay systems employing immobilised binding agents are a valuable tool in probing liquid samples.

Of particular interest are microarrays. (As used herein, the term “microarray” is intended to include nano-arrays. It will be understood that the term typically also includes mesoarrays.) Typically, two-dimensional microarrays are formed on substrates which have analyte binding agents immobilised on the substrate surface. Suitable analyte binding agents include proteins/peptides, nucleic acid molecules, lipids, viruses, tissues and cells. In microarray technology, the immobilised binding agents are provided on the surface of the substrate in discrete array elements (e.g. spots). The functionalised substrate is brought into contact with a liquid sample, e.g. a biological sample. Analytes in the solution may bind to spots of immobilised binding agent. The binding of analyte to the spots is detected, typically using fluorescently labelled reagents.

The liquid samples probed can have high complexity and include numerous components. For example, a biological liquid sample may be a tissue lysate from a patient biopsy. Different spots of the same microarray may have different immobilised binding agents, for example to determine the presence or absence of multiple different analytes in the liquid sample.

Multi-analyte microspot immunoassays were first described in the 1980s (see Ref. 1). DNA microarrays were first described in 1995 (see Ref. 2). Protein-protein interaction arrays are the subject of much interest at present. Proteomics and genomics applications of arrays are also of interest (see Refs. 3 to 6).

The fields of spot deposition and imaging techniques are fairly mature. Many different designs of surface chemistry for arrays have been produced, and are available to buy off the shelf, or can be created as needed in labs.

Microarrays typically occupy an area having dimensions no bigger than those of a standard microscope slide. The array elements (e.g. spots) of immobilised binding partner typically have a diameter of a few microns. They are typically positioned in square grids with array element spacings in the order of one hundred microns. Smaller array elements, on the nano-scale, are obtainable using techniques such as dip-pen nanolithography (see Refs. 7 to 9 and 14). This scaling down of array element size permits a reduction in the lateral dimensions of arrays, with the advantage of reducing the volumes of liquid sample interrogated.

Commonly, liquid samples are confined to the array region using a “frame”, which acts as a sealing gasket. This principle is employed both in manual arrays and automated systems. Examples include “FAST Frame” available from Whatman and “SIMplex” available from GenTel Biosciences Inc. Both frames generally require a relatively large sample volume (a minimum of about 80 μl and 70 μl respectively). In practice, leaks around the sealing gasket can cause cross-contamination. Holding the sealing gasket more firmly against the array slide substrate may reduce leakage, but can be detrimental to the integrity of the array surface. A similar technology is described in W02005/113131. A related system is the “ProPlate” array system available from Grace BioLabs. This system employs a frame comprising a plurality of bottomless wells which is bonded to the surface of an array slide, to provide a plurality of wells. Disadvantages of each of these “frame” systems include the potential for leakage, damage to the array or slide surface, evaporation of the liquid sample, and the requirement for relatively large sample liquid volumes.

A surface modification system called SlideImprinter, available from The Gel Company, addresses some of these problems. The Slideimprinter device prints warmed wax directly onto an array slide, to create hydrophobic boundaries around the wells. The 16-well design requires about 60 μl of sample liquid. Sample liquid evaporation remains a problem with this system. This system is relatively expensive, and requires modification of array slides once they have been made. This can be inconvenient and reduces the suitability of this system to scaled-up applications, for example in high throughput, automated systems. A similar system is described in

An alternative approach to reducing the required sample liquid volume involves reducing the vertical dimensions of microarray probing wells. For example, microarray probing can be carried out in a thin capillary, where about 120 μl of liquid sample is trapped under a slip over an array slide. The slip may be a plastic, planar slip such as the HybriSlip™, available from Grace Bio-Labs Invitrogen. A recent development is the LifterSlip™, available from Thermo Scientific and Erie Scientific, which is a glass slip which has two strips provided on the underside of the slip, to lift the slip slightly from the surface of the array slide. However, it is difficult to control sample spreading on the array slide, and the loading of the sample. This can result in air bubbles becoming trapped under the slip, reducing the reliability of the assay. The uncontrollability of sample spreading and loading can also result in increased “noise”, which reduces the sensitivity of the assay by increasing the minimum amount of analyte required for detection. Evaporation of the small volume of sample liquid before the slip is positioned can be a disadvantage in some contexts. Furthermore, it would be advantageous to provide still further reduction in the required sample liquid volume.

### SUMMARY OF THE INVENTION

The present inventors have realised that one or more of the above problems with the prior art can be addressed, overcome or reduced by providing an assay system having a first surface which has been patterned to define one or more “wells” e.g. by providing hydrophilic areas defined by hydrophobic boundaries, and a second surface, intended to be opposed to the first surface, having analyte binding agent (e.g. in a microarray) disposed on it. In this system, the sample liquid to be tested is disposed between the two surfaces, and contacts both. This can reduce problems associated with sample evaporation. The patterning confines the sample liquid to the hydrophilic regions. This confinement is believed to result from the surface tension of the liquid. The confinement of the sample liquid to the hydrophilic region(s) allows greater control of sample spreading, and reduces or avoids the formation of bubbles between the two surfaces. Providing two surfaces, one having the well patterning and the other supporting the binding agent, allows increased adaptability of the system, making it convenient both for manual laboratory assaying and in high-throughput (e.g. automated) systems.

Accordingly, in a first aspect, the present invention provides an assay assembly comprising
a first surface having at least one sample liquid wettable surface region defined by a sample liquid repelling boundary; and
a second surface, opposed to the first surface, having analyte binding agent immobilised thereon.

In this way, when a sample liquid is provided between the first and second surfaces, the liquid sample may be confined to the sample liquid wettable surface region(s), for example by the surface tension of the liquid. Accordingly, the liquid sample contacts region(s) of the second surface which are aligned with (e.g. overlie) the sample liquid wettable surface region(s) of the first surface. Conveniently, this allows very small volumes of liquid sample to be analysed.

Typically, the immobilised binding agent is provided on the second surface in one or more two-dimensional arrays of discrete array elements, e.g. wherein at least two different array elements of the microarray comprise different immobilised binding agent. It will be understood that the immobilised binding agent typically remains attached to the second surface under assay conditions, e.g. on contact with the sample liquid. In this way, analyte captured from the sample liquid by the binding agent is held on the second surface by the binding agent.

Some documents have discussed the use of microfluidics technology in biological applications. For example, reference 10 describes the formation of a liquid channel, by providing hydrophilic and hydrophobic regions on a substrate, with an overlying hydrophobic layer. Deposition of biological material from liquid in the channel is described. The deposition of the material was confined to areas in contact with the liquid in the channel.

Reference 11 describes the use of microfluidics chips to move droplets on a substrate, using electrochemistry. Biological applications of this technology are described, for example moving a sample droplet (e.g. containing glucose) into contact with a reagent droplet (e.g. containing glucose oxidase).

Reference 12 is concerned with merging microfluidics with microarray technology. It describes the formation of a network of fluidic channels, which can have immobilised binding agents on their interior. This work is contrasted with microarrays formed on a surface (e.g. two-dimensional microarrays).

In a second aspect, the present invention provides a kit of parts comprising:
a first surface having at least one sample liquid wettable surface region defined by a sample liquid repelling boundary; and
a second surface, having analyte binding agent immobilised thereon.

Conveniently, the first surface may be a surface of a first substrate, and the second surface may be a surface of a second substrate. It will be understood that the first and second surfaces are positionable such that the second surface is opposed to the first surface.

Preferably, the kit of parts further comprises a holder having a first substrate receiving site for holding the first substrate and a second substrate receiving site for holding the second substrate. The first and second substrate receiving sites are arranged or arrangeable to hold the first and second substrates in a position wherein the second surface is opposed to the first surface. For example, the first and second substrate receiving sites may be moveable relative to each other.

It will be understood that the first surface of the present invention may be used with numerous different second surfaces, for example second surfaces having different analyte binding agents immobilised thereon. For example, the first surface may be used in a first assay, washed, and then employed in another, different assay. It will be understood that the first surface may be supplied separately from the second surface. Accordingly, in a third aspect, the present invention provides a kit of parts comprising
a first substrate having a first surface, wherein the first surface has at least one sample liquid wettable surface region defined by a sample liquid repelling boundary; and
a holder having a first substrate receiving site for holding the first substrate and a second substrate receiving site for holding a second substrate,
wherein the first and second substrate receiving sites are arranged or arrangeable to hold the first and second substrates in a position wherein a surface of the second substrate is opposed to the first surface.

It will further by understood that the holder may be supplied separately from the substrates. Accordingly, in a fourth aspect the present invention provides a holder having a first substrate receiving site for holding the first substrate and a second substrate receiving site for holding a second substrate, wherein the first and second substrate receiving sites are arranged or arrangeable to hold the first and second substrates in a position wherein a surface of the second substrate is opposed to the first surface.

In a fifth aspect, the present invention provides a method of determining one or more properties of a sample liquid, the method comprising:
providing a sample liquid between a first surface and a second surface, wherein the first surface has at least one sample liquid wettable surface region defined by a sample liquid repelling boundary, and wherein the second surface is opposed to the first surface and has analyte binding agent immobilised thereon; and
determining one or more properties of the liquid sample by detecting binding of analyte to the immobilised analyte binding agent.

It will be understood that the sample liquid contacts the sample liquid wettable surface region (of the first surface) and the second surface. The sample liquid may be held between the two surfaces by the surface tension of the sample liquid.

In a further aspect, the present invention provides a method of making a first surface and a second surface, for example as defined in any of the aspects described herein. The method may comprise patterning a first surface to provide at least one sample liquid wettable surface region defined by a sample liquid repelling boundary. The method may comprise immobilising binding agent on a second surface. Preferably, the first surface is a surface of a first substrate. Preferably the second surface is a surface of a second substrate. The method may further comprise assembling an assay assembly by positioning the second surface in a position opposed to the first surface.

Typically, the sample liquid will be aqueous. It will be understood that the sample liquid wettable surface region may be a hydrophilic surface region. Similarly, the sample liquid repelling boundary may be a hydrophobic boundary. The sample liquid wettable surface region(s) may be referred to herein as hydrophilic surface region(s). Similarly, the sample liquid repelling boundary may be referred to as a hydrophobic boundary. As context allows, references to the hydrophilic surface region should be interpreted as references to the sample liquid wettable surface region, and references to the hydrophobic boundary should be interpreted as references to the sample liquid repelling boundary.

Further preferred and/or optional features of the invention will now be set out. It will be understood that the preferred and/or optional features described herein are applicable, either singly or in any combination, with any aspect of the invention, unless the context demands otherwise.

Preferably, the first surface is substantially planar, The patterning to provide the hydrophilic surface regions and the hydrophobic barriers may provide some unevenness on the first surface, and accordingly, it is understood that the first surface is preferably macroscopically planar, e.g. substantially flat. Preferably, each sample liquid wettable surface region is substantially planar.

Preferably, the contact angle of sample liquid with the sample liquid wettable surface region is 60° or less, for example 50° or less, 40° or less, 30° or less, 25° or less, or 20° or less. Preferably the contact angle of sample liquid with the sample liquid repelling boundary is at least 55°, at least 60°, at least 65°, at least 70°, at least 80°, or at least 85°. Preferably, the contact angle of sample liquid with the sample liquid repelling boundary is at least 5° greater than the contact angle of sample liquid with the sample liquid wettable surface region, for example at least 10° greater, at least 15° greater, at least 20° greater, at least 25° greater, at least 30° greater, at least 40° greater, at least 50° greater or at least 60° greater.

It will be understood that the contact angle is not a property inherent to the materials constituting the sample liquid wettable surface region and the sample liquid repelling boundary, but depends on the nature of the sample liquid. However, preferably the contact angles given above are the typical contact angles with the sample liquid intended to be used with the assay assembly. For example, the above contact angles may be typical contact angles with an aqueous sample liquid, or with water.

Preferably, the sample liquid wettable surface region(s) are provided by areas on the first surface constituted by a sample liquid wettable material, e.g. in a layer, for example a layer formed on the first substrate. Alternatively, the substrate itself may be made from sample liquid wettable material, and regions of the substrate may be exposed to form the sample liquid wettable surface region(s).

It will be understood that the nature of the material is not particularly limited, and depends on the intended sample liquid. Preferably, the material is hydrophilic. Suitable hydrophilic materials include oxides and nitrides, such as those employed in the semiconductor industry. Suitable oxides and nitrides include silicon dioxide, indium tin oxide and silicon nitride. Other suitable hydrophilic materials include glass, such as soda lime glass, borosilicate glass, and fused quartz. Further suitable hydrophilic materials are hydrophilic polymers such as acrylates (e.g. methacrylate), acrylamides (e.g. methacrylamide) and polyalcohols, (e.g. polyvinyl alcohol), and hydrogels.

Preferably, the sample liquid repelling boundary or boundaries are provided by area(s) of sample liquid repelling material, e.g. in a layer, for example a layer formed on the first substrate. Alternatively, the substrate itself may be made from sample liquid repelling material, and regions of the substrate may be exposed to form the sample liquid repelling boundary.

It will be understood that the nature of the material is not particularly limited, and depends on the intended sample liquid. Preferably, the material is hydrophobic. Suitable hydrophobic materials include fluoropolymers (e.g. amorphous fluoropolymers) such as Teflon® from Du Pont®, CYTOP® from Asahi Glass, Fluoropel® from Cytonix (http://cytonix.com/fluoropel.html) or polymerised C₄F₈, or parylene (e.g. parylene C). Alternatively, the hydrophobic material may be wax, or a hydrocarbon polymer such as polyethylene, polypropylene and polybutylene. Still further suitable hydrophobic materials include siloxanes, such as polydimethyl siloxane.

The material of the first substrate is not particularly limited. The first substrate may be made of silicon. For example, it may be a silicon wafer. Alternatively, it may be made of glass, such as soda lime glass, borosilicate glass, or it may be made of fused quartz. In this case, the hydrophilic surface region(s) may be provided by exposed regions of the substrate. For example, the substrate may be a microscope slide. As a further alternative, the first substrate may be made of polymer. For example, it may be made from hydrophobic or hydrophilic polymer, for example the hydrophobic and hydrophilic polymers described herein.

The pattern of hydrophilic area(s) defined by hydrophobic boundaries may be formed by any suitable method. One particularly suitable method is etching. Etching is well known, for example in the semiconductor manufacturing industry. The etching method may comprise the following steps:

(i) forming a first layer on the substrate (e.g. a layer of hydrophobic material or a layer of hydrophilic material);
(ii) forming a second layer on top of the first layer (where the first layer is hydrophobic, preferably the second layer is hydrophilic, and vice versa);
(iii) forming a resist layer (e.g. a photoresist layer) on top of the second layer;
(iv) removing portions of the resist layer to expose areas of the second layer (sometimes referred to as patterning the resist);
(v) etching the exposed areas of the second layer to expose areas of the first layer; and
(vi) removing any remaining resist layer.

Where the substrate itself is to be exposed to provide the sample liquid wettable surface region(s) or the sample liquid repelling boundary, the step of forming a first layer may be omitted. In that case, references to the first layer may be interpreted as references to a surface of the substrate. For example, where the substrate is made from hydrophilic material, a layer of hydrophobic material may be formed on the hydrophilic substrate, and then etched (e.g. steps (iii) to (vi) above) to expose regions of the hydrophilic substrate.

The first layer may be an oxide layer. For example, it may be formed by oxidation (e.g. thermal oxidation) of a surface of the first substrate. Alternatively, the first layer may be formed by plasma enhanced chemical vapour deposition. Other suitable methods of forming the first layer, e.g. a layer of sample liquid wettable material, will be apparent to the skilled person. A suitable thickness for the first layer is about 0.5 μm to about 1 μm.

The second layer should preferably be deposited substantially evenly over the surface of the first layer. After it is formed, it should preferably be substantially free of gaps exposing the first layer (although of course portions of the first layer may be exposed later in the process). Suitable methods for depositing the second layer include solution spinning (e.g. for Teflon® and CYTOP®), deposition from vapour (e.g. for parylene) and deposition from plasma (e.g. for C₄F₈).

Suitable methods of depositing resist layers are well known. The resist layer may be patterned using a mask, for example by exposing the resist layer to radiation (e.g. UV radiation) through a mask. Surfactant may be employed to enhance resist adhesion to the second layer. This is particularly useful when the second layer is strongly hydrophobic, but is not necessary in the case of parylene.

The exposed areas of the second layer may be etched by exposure to oxygen plasma. After this, any remaining resist may be removed, for example by dissolving the resist in acetone.

Alternatively, the patterning of sample liquid wettable surface region(s) and sample liquid repelling boundaries may be provided by modification regions of the first surface. For example, where the first surface is a hydrophobic material (e.g. a hydrophobic polymer), regions of the hydrophobic material may be treated to make it more hydrophilic, in order to provide the hydrophilic region(s). For example, hydrophilic regions may be provided by plasma oxygen treatment of hydrophobic material or by functionalising regions of the hydrophobic material with hydrophilic functional groups such as —OH and/or —COOH.

As a further alternative, sample liquid wettable surface regions may be formed by grafting or depositing sample liquid wettable material onto a sample liquid repelling surface or substrate, or sample liquid repelling boundaries may be formed by grafting or depositing sample liquid repelling material onto a sample liquid wettable surface or substrate. This option is particularly suitable where both the sample liquid wettable material and the sample liquid repelling material are polymers.

As described above, the first surface has at least one sample liquid wettable surface region defined by a sample liquid repelling boundary. Preferably, the first surface includes a plurality of sample liquid wettable surface regions, each defined by a sample liquid repelling boundary. For example, the first surface may have 16 liquid wettable surface regions each defined by a sample liquid repelling boundary. This arrangement may be preferred as microarray slides typically include 16 microarray areas (i.e. “array blocks”, or “sub-arrays”).

Preferably, each sample liquid wettable surface region has a two-dimensional shape. Preferably, this two-dimensional shape has suitable dimensions for the sample liquid wettable surface region to contact a two-dimensional microarray. For example, the shape may have at least two dimensions of at least 0.5 mm, more preferably at least 1, 2, 3, 4, 5 or 6 mm. Said at least two dimensions may be perpendicular dimensions. It may be preferred that each sample liquid wettable surface region has a substantially polygonal shape, optionally with rounded corners and/or with overflow regions as described below. For example, each sample liquid wettable surface region may be substantially rectangular (e.g. substantially oblong or, more preferably, substantially square), or substantially hexagonal. It will be understood that each sample liquid wettable region may have curved edges. For example, each region may be substantially circular, substantially oval or substantially elliptical. (Herein, the terms used to refer to particular shapes should not be interpreted as limited to the strict mathematical meaning of these words).

Typically, each sample liquid repelling boundary has an elongate shape. Preferably, each sample liquid wettable surface region has a width greater than its sample liquid repelling boundary, for example at least 1.1 times, at least 1.2 times, at least 1.3 times, at least 1.5 times, at least 2 times or at least 3 times the width of its sample liquid repelling boundary.

It may be preferable that the shape of one or more of the sample liquid wettable surface regions provides one or more overflow regions. These allow for small inaccuracies of the volume of sample liquid provided. Where the sample liquid wettable surface region is substantially polygonal, these overflow regions may be located at the corners of the polygon. This can help to ensure that sample liquid flows right into the corners of the sample liquid wettable surface regions, and accordingly can help to maximise the area of contact of the sample liquid with the second surface. For example, it can help to ensure that the sample liquid contacts the entire area of a microarray provided on the second surface.

The first surface may be provided with one or more inlet channels, for loading sample liquid into the sample liquid wettable surface region(s). Each inlet channel is in fluid communication with at least one, and preferably only one, of the sample liquid wettable surface regions, The channel may provide a path for sample liquid from the edge of the first substrate to the sample liquid wettable region. Alternatively, the channel may provide a path for sample liquid from an area of the first surface which is exposed (e.g. not covered by the second surface) towards the sample liquid wettable region (which may be covered by the second surface).

The channels may be elongate areas of sample liquid wettable surface material, defined by a sample liquid repelling boundary. Alternatively, the channel may be a physical groove on the first surface. Each channel may be adapted to draw sample liquid through the channel and into the sample liquid wettable surface region by capillary action. Alternatively, the channel may be adapted to receive a tube or pipette tip for sample loading. Providing inlet channels can be particularly advantageous, as it allows sample liquid to be loaded after the first and second surfaces are in position. This can further reduce sample liquid evaporation, provide control of sample spreading and reduce or avoid bubble formation.

Alternatively, of course, sample liquid may be loaded onto the first surface, and then the second surface may be lowered on top. Even in such embodiments, the control of sample spreading can be improved, and the formation of bubbles reduced, due to the confinement of sample liquid in the sample liquid wettable surface region(s). As described in more detail below, the holder can provide control of second surface lowering.

In some embodiments, the overflow region may be provided by the inlet channel. The may be preferred, for example, when a single sample liquid wettable surface region is provided on the first surface. However, in other embodiments, e.g. where multiple sample liquid wettable surface regions are provided, it may be preferred that the one or more overflow regions are distinct form the inlet channel. For example, one or more overflow region may be provided at a position distinct from the inlet channel.

Preferably, the second surface is substantially planar. The immobilised binding agent may provide some unevenness on the second surface, e.g. resulting from pillars or clusters of cells or particles. Accordingly, it is understood that the second surface is preferably macroscopically planar, e.g. substantially flat.

The immobilised binding agent may be provided in an array of discrete array elements (e.g. spots) of immobilised binding agent. The array elements may typically have a diameter of about 100 μm or less. The spacing between array elements may be about 100 μm or less. Preferably, the array elements are provided in a two-dimensional array on the second surface. The elements of the array may each comprise the same binding agent. Alternatively, different array elements may comprise different binding agents, so that the array includes array elements of at least 2 different binding agents. The array may include 16 or more, for example 25 or more, or 50 or more discrete array elements. Preferably, each array is substantially planar (e.g. macroscopically planar such as substantially flat).

It will be understood that the array(s) may comprise control elements which provide positive or negative controls, for example to confirm satisfactory sample wetting and/or satisfactory analyte binding.

The second surface may comprise multiple arrays of discrete array elements. For example, the second surface may comprise the same number of arrays as the number of sample liquid wettable surface regions on the first surface. For example, where the second surface has 16 arrays, preferably the first surface has 16 sample liquid wettable surface regions. Preferably, each array is substantially planar. Preferably, the second surface is arranged so that each array is aligned with (e.g. overlies) a sample liquid wettable surface region of the first surface.

It may be preferred that the first and/or second substrate is transparent or translucent. For example, it may be a glass substrate such as a glass microscope slide. This allows the distribution of sample liquid between the first and second surfaces to be readily observed. This may also allow detection of analyte binding to be carried out without dismantling the assembly. In some preferred embodiments, one or more of the discrete array elements comprise a visualisation component, which allows one or more properties of the liquid sample to be determined by detecting binding of analyte to discrete array elements of immobilised analyte binding agent.

It will be understood that the nature of the immobilised binding agent(s) is not particularly limited in the present invention. Suitable binding agents will be well known to those skilled in the art. For example, the immobilised binding agent(s) may be selected from proteins/peptides, antibodies, receptors, nucleic acids such as DNA or RNA, lipids, carbohydrates, lipid-bound proteins, glycoproteins, glycolipids, aptamers, haptens, antigens, small molecules, whole cells such as bacterial cells, and viruses.

Methods of providing one or more microarrays on the second surface will be well known to those skilled in the art. For example, dip-pen nanolithography may be employed, or other spotting using commercially available arrayer tips, e.g. piezo-dispenser tips.

The second surface may include at least one sample liquid wettable region defined by a sample liquid repelling boundary, similarly to the first surface. Accordingly, it will be understood that the features of the first surface described above may apply equally the second surface, singly or in any combination. In embodiments wherein the second surface includes at least one sample liquid wettable region defined by a sample liquid repelling boundary, preferably the pattern of sample liquid wettable surface region(s) and sample liquid repelling boundaries on the second surface mirrors that of the first surface, in order that the sample liquid wettable surface region(s) of the second surface are aligned with (e.g. overlie) the sample liquid wettable surface region(s) of the first surface, and in order that the sample liquid repelling boundaries of the second surface are aligned with (e.g. overlie) the sample liquid repelling boundaries of the first surface. This can further improve sample liquid confinement. It will be understood that immobilised binding agent should be positioned on the sample liquid wettable surface region(s) of the second surface.

Preferably, a sample liquid volume of 80 μl or less is required for each sample liquid wettable surface region. More preferably, a sample liquid volume of 50 μl or less, 40 μl or less, 30 μl or less, 25 μl or less, 20 μl or less, or 15 μl or less, or 10 μl or less, or 5 μl or less is required. Preferably, the dimensions of each sample liquid wettable region, and the gap between the first and second surfaces define a sample liquid zone having a volume of 60 μl or less, more preferably 50 μl or less, 40 μl or less, 30 μl or less, 25 μl or less, 20 μl or less, or 15 μl or less, or 10 μl or less, or 5 μl or less. Of course, it will be understood that larger sample volumes may be used, for example if the gap between the first and second surfaces is increased, or if the gap between the first and second layers is increased. A sample volume of 250 μl or less may be preferred in some cases.

It will be understood that the volume of sample liquid required depends on the area of the sample liquid wettable surface region. Preferably, the volume of sample liquid required for each sample liquid wettable surface region is 0.4 μl or less per mm² of sample liquid wettable surface region, more preferably 0.3, 0.2 or 0.1 μl or less per mm² of sample liquid wettable surface region. Typically, the volume of sample liquid required for each sample liquid wettable surface region is at least 0.050 per mm² of sample liquid wettable surface region. Typically, the space between the first surface and the second surface is about 200 μm. For example, it may be 500 μm or less, 400 μm or less, 300 μm or less, 200 μm or less, or 100 μm or less. Typically, the space between the first surface and the second surface is at least 10 μm, at least 50 μm, or at least 100 μm.

A holder may be provided, having a first substrate receiving site for holding the first substrate, and a second substrate receiving site for holding the second substrate. (As described above, the first and second surfaces may be provided on first and second substrates respectively.) The first and second substrate receiving sites may be arranged or arrangeable to hold the first and second substrates in a position wherein the second surface is opposed to the first surface. Providing a holder can help to control the relative positioning of the first and second surfaces, for example helping to align the sample liquid wettable surface regions with immobilised binding agent (e.g. with an array) on the second surface.

It will be understood that preferably one or both of the substrate receiving sites are configured to restrict (e.g. substantially prevent) lateral movement of the substrate with respect to the holder. For example, one or both of the substrate receiving sites may comprise a recess having a side wall or walls which are arranged to restrict (e.g. substantially prevent) lateral movement of the substrate with respect to the holder. In this way, the relative orientation of the first and second substrates may be controlled, for example to ensure suitable alignment between regions of the first and second surfaces as described above. One or more biasing elements may be provided to assist in holding the first and/or second substrate in their respective substrate receiving sites. The biasing element may be arranged to bias the substrate towards a side wall or walls, for example an opposing side wall or walls, in order to hold the substrate against the side wall or walls. The biasing element may comprise a spring or other resilient member.

One or more of the substrate receiving sites may be provided with a stopper to restrict removal of the substrate from the substrate receiving site. For example, the stopper may be a clip, which may be resiliently biased to a position or arrangement in which it restricts or prevents removal of the substrate from the receiving site. For example, the clip may comprise a protrusion resiliently biased towards a position in which it overlies the substrate. However, it will be understood that the first and/or second substrates are preferably removable from the holder, to allow washing and further analysis.

The first and second substrate receiving sites may be moveable relative to each other. In this way, the second surface may be moved towards the first surface in a controlled manner, allowing good control of sample spreading, and reducing the formation of bubbles. The holder may be configured to allow the second substrate to be separated from the first substrate while keeping the second surface substantially horizontal. This can help to reduce cross-contamination of sample liquids which contact different regions of the second surface. For example, the first and second substrate receiving sites may be connected by a hinge.

One or more spacer components may be provided, as part of the holder, as part of the first substrate, as part of the second substrate or separately. The spacer components are used to provide a space between the first and second surfaces. The spacer components may have adjustable height, to allow adjustment of the gap between the first and second surfaces. For example, the spacer components may be screws, which can be raised or lowered to adjust the gap between the first and second surfaces. Alternatively, the spacer components may be blocks or pillars which are positionable between the first and second surfaces. Blocks of different heights may be provided, or multiple blocks may be stacked, in order to adjust the gap between the first and second surfaces.

The holder may be provided with one or more spacer receiving sites for holding a spacer. Preferably, the spacer receiving sites restrict (e.g. substantially prevent) lateral movement of the spacer with respect to the holder. For example, the holder receiving site may comprise a recess having a side wall or walls which area arranged to restrict (e.g. substantially prevent) lateral movement of spacer. In this way, the relative orientation of the spacer and the first and/or second substrates may be controlled. One or more biasing elements may be provided to assist in holding the spacer(s) in their respective spacer receiving sites, as described above with respect to the substrate receiving sites.

Preferably, where one or more spacers is provided, the spacers are positioned to contact the first surface at a point which is not part of a sample liquid wettable region. Similarly, preferably the spacers are positioned to contact the second surface at a part of the surface which does not have immobilised binding agent immobilised thereto, e.g. at a part of the surface which does not have an array formed thereon. Preferably, the one or more spacer receiving sites are positioned accordingly. A stopper may be provided to restrict removal of the spacer from the spacer receiving site. The stopper may have the features described above with reference to the stopper for restricting removal of the substrate from the substrate receiving site.

The holder may be closable, in order to encase the first and second substrates. Accordingly, the holder may have an open configuration to allow the first substrate, second substrate and/or spacer components to be loaded into the holder, and a closed configuration in which the first substrate, second substrate and/or spacer components are held within the holder. The holder may comprise a locking element, for keeping the holder in its closed configuration.

The holder may comprise one or more compressor elements, for urging the first substrate towards the second substrate, and/or for urging the second substrate towards the first substrate, e.g. when the holder is in its closed configuration. Preferably, the compressors are provided at positions corresponding to the spacers (or spacer receiving sites). By providing pressure only at these positions, a highly parallel sandwich, without buckling or tension, is provided. Preferably, the compressors are sprung. This can help to avoid application of excessive pressure to the substrates.

The holder may be configured so that one or both of the first and second substrates are visible when the holder is in its closed configuration. This may allow the sample loading and the distribution of sample liquid between the first and second surfaces to be readily observed. This may also allow detection of analyte binding to be carried out without dismantling the assembly.

Preferably, the holder provides access to the inlet channels for ample loading, e.g. when the holder is in the closed position. In this way, the assay may be assembled and the first and second substrates held in place, before sample liquid is loaded via the inlet channels. Access to the inlet channels may be provided, for example, by a window or other opening in the holder.

As described above, the present invention provides a method of determining one or more properties of a sample liquid. It will be understood that this method may be carried out using the assay assembly of the invention.

The sample liquid may be provided to the assay assembly by any suitable method. Preferably, the sample liquid is provided via an inlet channel. In this way, sample liquid may be supplied after the second surface is positioned opposed to the first surface. The sample liquid may be transferred to the sample liquid wettable surface region by capillary action. Alternatively or additionally, force from the sample liquid dispenser (e.g. pipette) may urge the sample liquid towards the sample liquid wettable surface region. Movement of the sample liquid from an inlet channel into the sample liquid wettable surface region can improve the assay, as it can enhance movement of the sample liquid relative to the immobilised binding agent.

Alternatively, the sample liquid may be dispensed directly onto the sample liquid wettable region, before the second surface is positioned opposed to the first surface (e.g. before the second surface is lowered onto the first surface).

The skilled person understands that assay quality can be improved by enhancing diffusion of analytes in the sample liquid relative to the immobilised binding agent (e.g. relative to an array). For example, diffusion of analytes may be enhanced by agitation, for example mechanical, electrical, or magnetic agitation, or by radiation.

The assembly may be encased in a chamber providing a suitable atmosphere for performing the assay. For example, the assembly may be encased in a humidity chamber.

The property of the sample liquid to be determined in the method of the present invention is not particularly limited. For example, the property may be the presence or absence of one or more analytes in the sample liquid. Alternatively, it may be the concentration of one or more analytes in the sample liquid. As a still further alternative, the property of the sample liquid may be a property of one or more analytes in the sample liquid. For example, the property may be the binding affinity of the analyte for the immobilised binding agent, or the size of the analyte.

Binding of the analyte to immobilised binding agent may be detected by any suitable method. Suitable methods will be well known to those skilled in the art. For example, binding may be detected using labelling, for example fluorescence labelling. For example, total internal reflection fluorescence may be used. Conveniently, binding may be detected with or without taking apart the assembly, depending for example on the type of detection employed, and the material of the assembly.

Reference 13, which is incorporated by reference herein in its entirety, describes microarray technology in some detail. In particular, it describes suitable binding agents (referred to as “capture probes” in that document). It also describes suitable labelling and detection systems.

As described above, the first surface may be used in a first assay, washed and then used in a second assay, for example a different assay using a different second surface. Preferably, in between each assay or probing step the first surface is washed. Preferably, at least the final wash step is carried out substantially in the absence of detergent or surfactant. Presence of detergent or surfactant may reduce the efficiency of sample liquid containment on the first surface. Preferably, the first surface is dried, e.g. in a gas stream such as a nitrogen or dry air stream, or by spinning, after washing. It will be understood that washing the first surface substantially in the absence of detergent is desirable also prior to the first use of the surface. Similarly, the drying step may be performed prior to the first use of the surface.

### BRIEF DESCRIPTION OF THE DRAWINGS

Preferred embodiments and examples of the invention will now be described with reference to the figures, in which:

FIG. 1 illustrates example arrangements of hydrophilic surface areas on the first surface.

FIG. 2 shows photographs of example assemblies with sample liquid loaded.

FIG. 3 illustrates an example method of making the first surface.

FIG. 4 illustrates example sample loading techniques.

FIG. 5 illustrates the adjustable gap between the first and second surfaces.

FIG. 6 shows satisfactory spreading of sample liquid on assemblies according to embodiments of the present invention.

FIGS. 7 and 8 show results of tests indicating satisfactory analyte diffusion in sample liquid on assemblies according to embodiments of the present invention.

FIGS. 9 and 10 show results from Example 3, and illustrate that cross-contamination was avoided, and that an even distribution of signal intensities was achieved.

FIG. 11 shows results of Example 4, where assemblies according to embodiments of the present invention were compared with a frame assembly.

FIG. 12 shows the results of Example 5, and shows array images and the coefficient of variation from an assembly according to an embodiment of the present invention (labelled “CHIP”), and LifterSlip.

FIG. 13 shows a preferred embodiment of the assay assembly of the present invention.

FIG. 14 shows a plan view of a portion of an assay assembly according to an embodiment of the present invention.

### DETAILED DESCRIPTION OF THE PREFERRED EMBODIMENTS AND FURTHER OPTIONAL FEATURES OF THE INVENTION

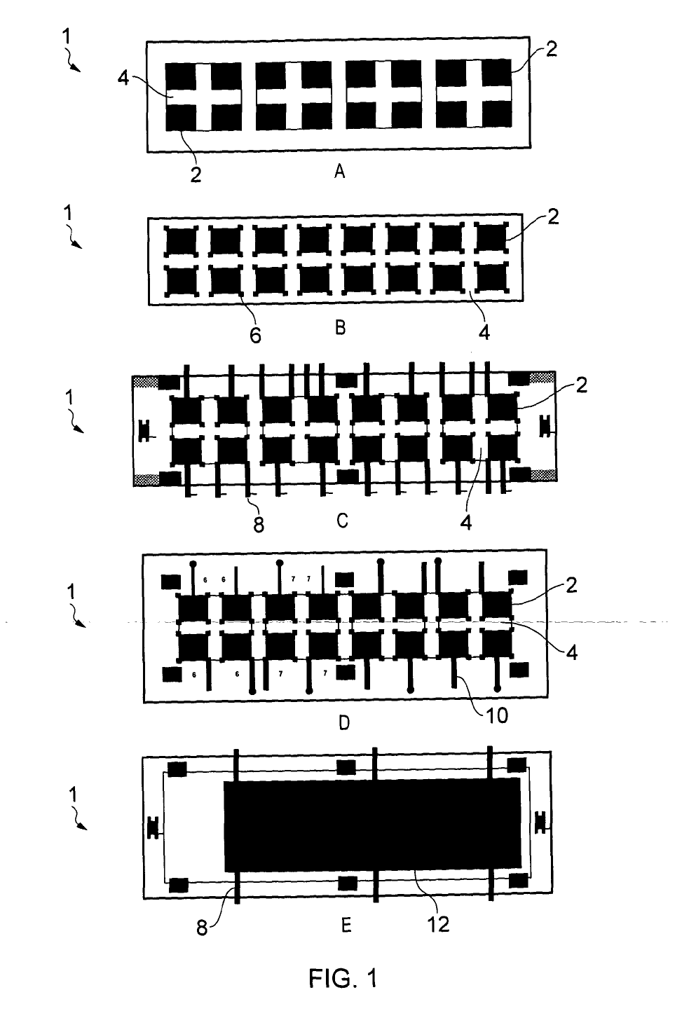

Preferred arrangement of the hydrophilic surface regions will now be described with reference to FIG. 1.

FIG. 1A shows a first surface 1 which has sixteen square shaped hydrophilic surface regions 2, defined by hydrophobic boundaries 4.

FIG. 1B shows an adaptation of the surface shown in FIG. 1A, where the hydrophilic surface regions 2 are each provided with overflow areas 6. The width of each square hydrophilic region is, for example, about 6 mm. The spacing from the left hand side of one hydrophilic region to the left hand side of the next is, for example, about 9 mm. The edges of the overflow areas are spaced approximately 0.5 mm from the edge of the main hydrophilic region.

FIG. 1C shows an adaptation of the surface shown in FIG. 1B, where channels 8 are provided, for loading sample liquid. The channels 8 extend to the sides of the surface. They are physical grooves, designed to accommodate a tube or pipette for loading the sample.

FIG. 1D shows an adaptation of the surface shown in FIG. 1B, where channels 10 are provided, for loading sample liquid. The channels 10 are regions of hydrophilic material, defined by hydrophobic boundaries. The channels 10 are intended to extend to an area of the first surface which, in use, is not covered by the second surface.

FIG. 1E shows a surface having a single hydrophilic surface region 12. Channels 8 are provided for loading the sample.

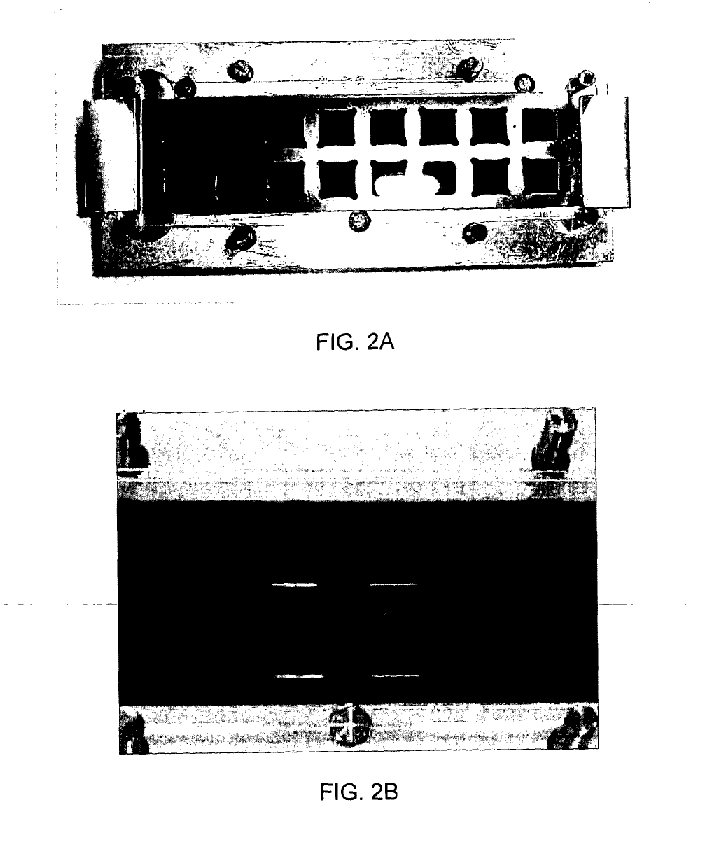

FIG. 2A shows a photograph of an example assembly with sample liquid loaded. The sample liquid has been dyed to improve its visibility. The sample liquid is Chromotrope 2R solution in PBS. Each of the wells contains 11 μl. The gap between the first and second surfaces is approximately 280 μm. FIG. 2B shows four wells filled with 4 μl of dye solution. The gap between the first and second surfaces is approximately 103 μm.

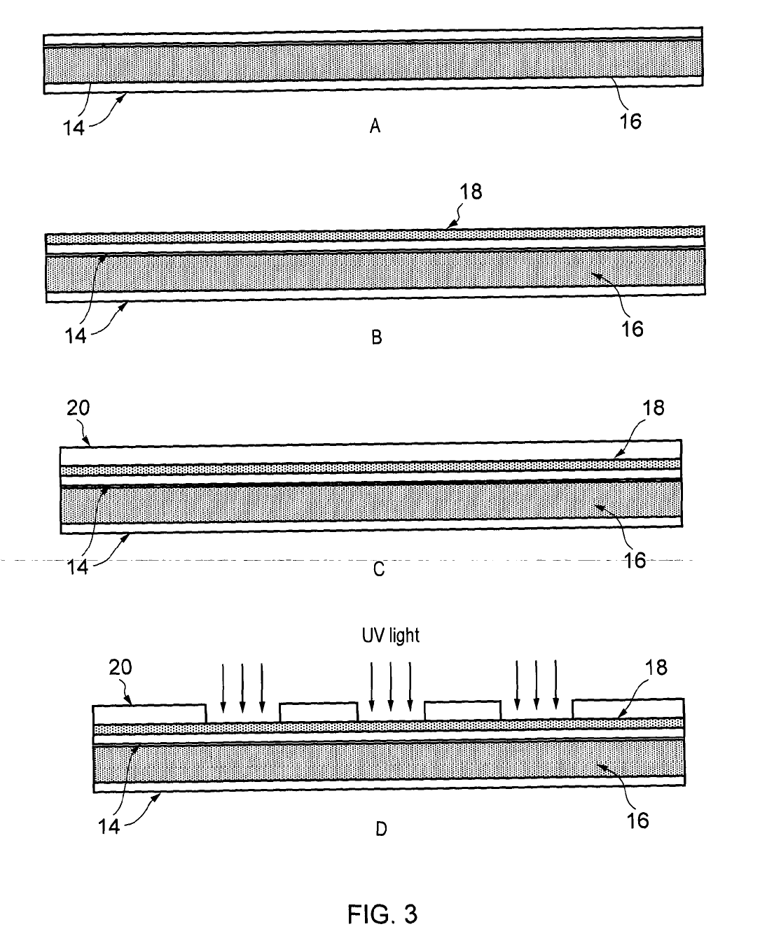

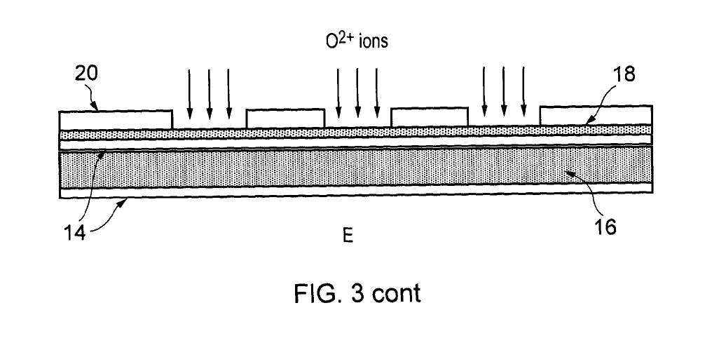

An example first surface manufacturing process will now be described, with reference to FIG. 3.

The first substrate is made of a silicon wafer 16, having a thickness of typically about 525 μm. The wafer is thermally oxidised in a furnace to generate a hydrophilic SiO₂ layer 14, having a thickness of approximately 0.5-1 μm, as shown in FIG. 3A. (Alternatively, plasma enhanced chemical vapour deposition or other method of forming a uniform oxide layer can be used.)

A hydrophobic layer 18, such as Parylene C or an amorphous fluoropolymer (aFP), e.g. Teflon®, CYTOP®, or polymerized C₄F₈, is deposited over the whole wafer, as shown in FIG. 3B. Thickness of the polymer deposited should be sufficient to avoid defects such as pinholes and ensure uniformity of the coating, but 20-100 nm is usually sufficient. Teflon and CYTOP are spun on from solution, Parylene C is deposited from vapour and C₄F₈ from electrical plasma. It will be understood that best confinement of liquid sample is achieved with the most hydrophobic layers

The hydrophobic layer 18 is patterned by spinning a layer of photoresist 20 on top of the hydrophobic layer as shown in FIG. 3C and exposing the photoresist layer 20 under a mask pattern to UV light, then developing, as shown in FIG. 3D. Because the aFP materials are very hydrophobic, surfactant may be added to the resist solution to promote adhesion, but this is not necessary in the case of Parylene.

In the next step the wafer 16 is exposed to an oxygen plasma in a vacuum system to etch the hydrophobic layer to expose hydrophilic regions as shown in FIG. 3E. Remaining resist is dissolved in acetone. Note that the etch rates of the resist and aFP are very similar, therefore the resist needs to be thick enough to survive the etch process.

The wafer may then be diced to size by a diamond saw, rinsed and dried ready to be mounted into the holder.

An example washing method for preparing the first surface for its first use, or for reconditioning it for re-use will now be described. The first surface is cleaned by rinsing it, e.g. using wash bottles in copious amounts of distilled water, followed by a rinse in NaOH (˜0.1M) solution, another rinse in distilled water, followed by a rinse in HCl (˜0.1M) solution and then rinsing in distilled water again. As a result of successful chip reconditioning, a droplet of water or a water-based solution spreads thinly and fills the hydrophilic pattern. Enzymes and/or surfactants may be applied to enhance sample removal.

When the 16-well chip has been adequately washed, after pouring water over the chip and shaking off excess, one can observe all the hydrophilic patches to contain similar amounts of water arranged in similar droplet shapes. The appearance of this pattern of liquid spreading makes it easy for the operator to judge when the chip has been adequately washed.

After washing the first surface is dried under a stream of dry nitrogen for approximately one minute, or until no bulk water is visible, and is ready for use.

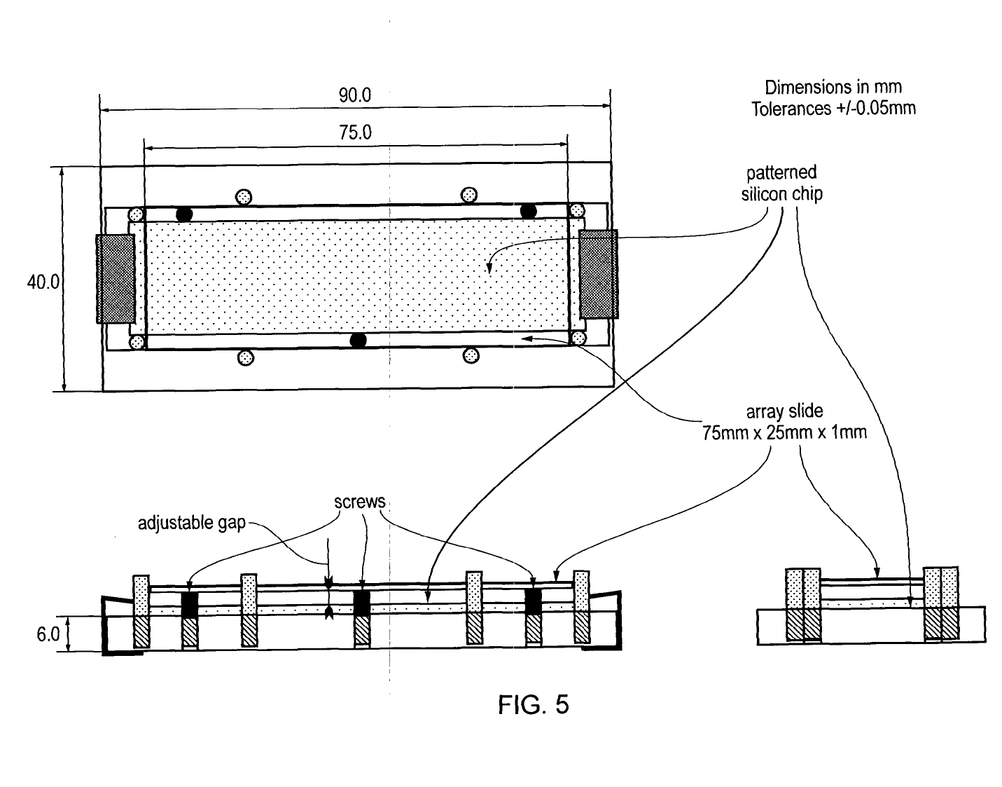

In embodiments wherein an adjustable spacer is provided, the following protocol may be used for adjusting the gap between the first and second surfaces. The spacers (e.g. screws) should be adjusted (e.g. raised) to a position in line with the tops of the liquid droplets. Then, a second surface, or a model second surface such as a clean plain glass slide is lowered onto the spacers and then the spacers are adjusted so that the pads of liquid take on the form of the hydrophilic region, e.g. a square shape. For 6×6 mm square regions, the liquid volumes may be as low as 2 μl (corresponding to gap set at about 50 μm) up to, for example 15-20 μl. This is illustrated in FIG. 5, which shows example dimensions of some components. In FIG. 5, the first substrate is referred to as “patterned silicon chip”, and the second substrate as “array slide”. The spacers are referred to as “screws”.

Example sample loading techniques will now be described.

In a first example technique, a desired volume of sample is carefully pipetted onto the hydrophilic regions of the first surface. (In case of small volumes, accuracy may be improved by ensuring that the pipette tip touches the hydrophilic surface regions. For example, for this ‘top-loading’ of volumes of 10 μl and less, a high specification pipette (in terms of both accuracy and precision, e. g. model EDP3 from Rainin, or an electronic pipette with LTS shaft) are recommended.

After the sample is loaded, the array slide (second surface) is slowly and steadily lowered onto the spacers, for example using wide flat wafer tweezers (for example, model 667. SA from Windrush Technology). Slow lowering and a very horizontal orientation of the array slide help to avoid trapping of bubbles and collapse of the top slide at the side of the assembly.

The sample will then spread into the shape defined by the hydrophilic region, the hydrophobic barrier, and the array slide (second surface).

Different samples of water-based solutions were loaded into the ‘wells’ in contact with a variety of different ‘array’ slides without cross contamination. In cases where the array slide is transparent or semi-transparent addition of a brightly coloured solution allows to take photographs of filled ‘wells’ under conventional room lighting to show separation of the wells, as shown for example in FIG. 2.

When the added solutions are not coloured the boundary of the ‘wells’, the sample spreading is still easily detectable by eye in ordinary lighting. Thus the present invention allows for direct verification of the shape and dimensions adopted by the sample liquid in contact with the array surface.

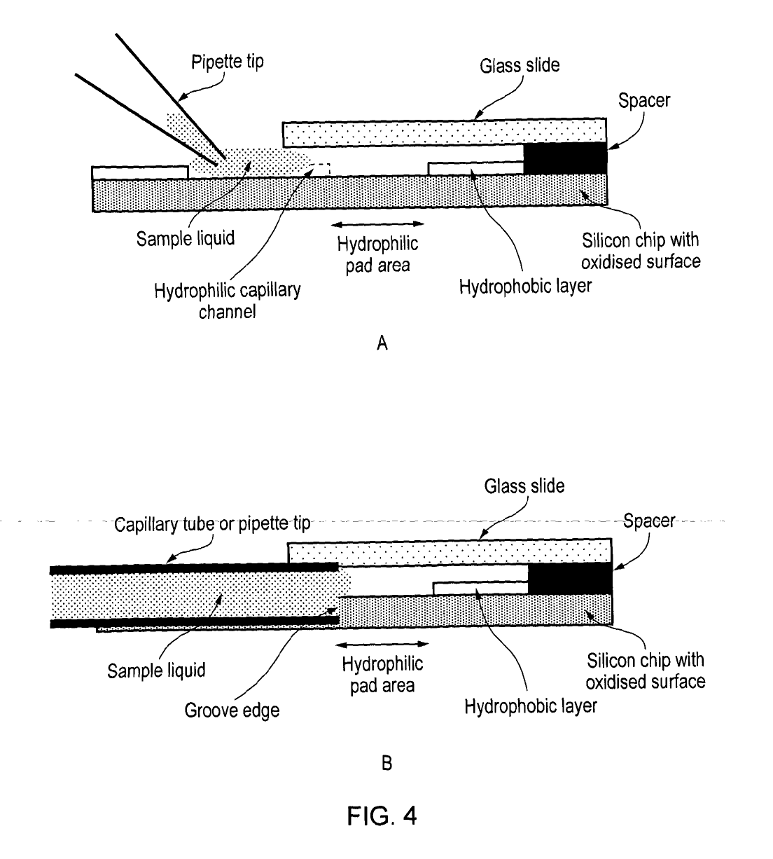

Alternatively, sample liquid can be loaded into the assembly using a “side loading” technique. Suitable techniques are illustrated in FIG. 4.

In FIG. 4A, sample liquid is loaded, from a pipette tip or capillary tube, onto an exposed (hydrophilic) region of the first surface (referred to in FIG. 4A as a silicon chip with oxidised surface). It passes along the hydrophilic capillary channel into the hydrophilic surface region (called hydrophilic pad area in FIG. 4A). The second surface is referred to as a glass slide in FIG. 4A. The liquid sample passes along the capillary channel and into the hydrophilic surface region. A spacer is provided between the first and second surfaces.

In FIG. 4B, sample liquid is loaded, from a pipette tip or capillary tube, directly onto the hydrophilic surface region, via a groove formed in the first surface. Otherwise, FIG. 4B is similar to FIG. 4A. Similar components are labelled similarly. This “groove loading” method may be particularly suitable when the sample liquid is a gel.

Alternatively, other methods such as surface acoustic waves, or electrowetting on dielectric, may be used to move the liquid sample to its desired position.

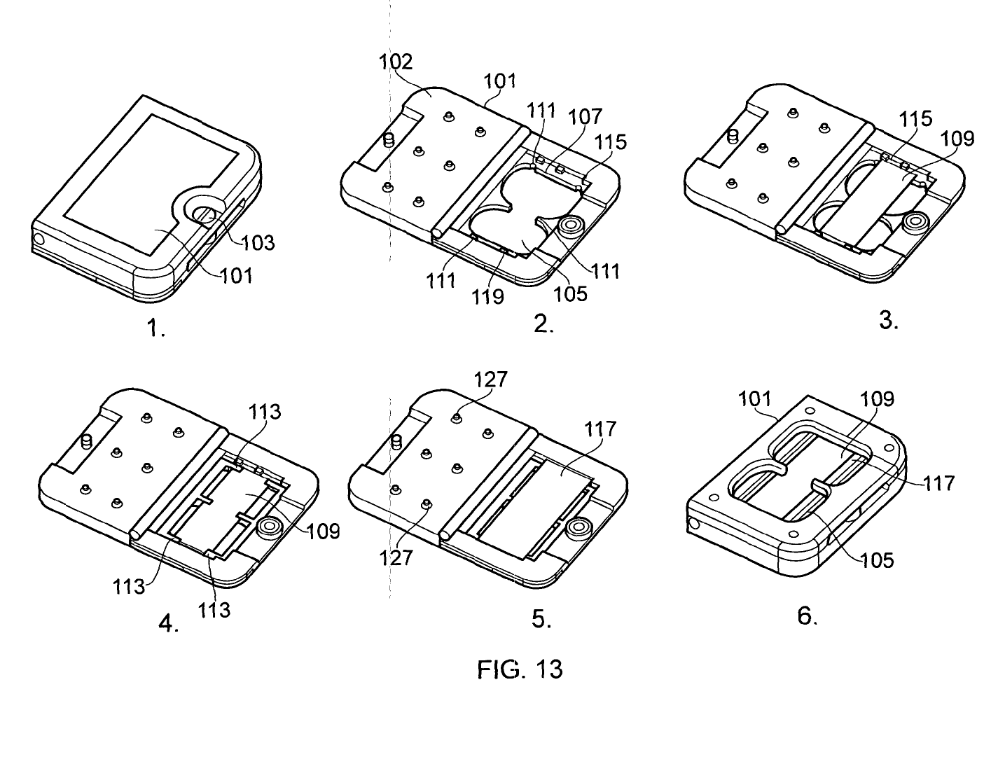

A preferred embodiment of the assay assembly will now be described with reference to FIG. 13. Image 1 of FIG. 13 shows the underside of the holder 101 in its closed configuration. A locking screw 103 is provided. Image 6 of FIG. 13 shows the upper side of the holder 101 in its closed configuration. Images 2 to 5 of FIG. 13 show the holder 101 in its open configuration, having a lid portion 102. A window 105 is provided in the holder casing, to allow the first and second substrates to be seen, and to allow for sample loading. The holder comprises a recess 107 for receiving a second substrate 109, which has a plurality of microarrays formed on its surface. The walls of the recess substantially prevent lateral movement of the second substrate 109 with respect to the holder 101. A biasing element 119 is provided for urging the second substrate 109 towards the opposite side walls of the recess 107. This biasing element 119 is provided in a corner of the recess 107 in order to urge the second substrate towards the two side walls which meet at the opposite corner of the recess 107.

Recesses 111 are provided for receiving spacers 113. The walls of the recesses substantially prevent lateral movement of the spacers 113 with respect to the holder 101 and the second substrate 109. A further recess 115 is provided for receiving the first substrate 117, which has a plurality of sample liquid wettable surface regions defined by sample liquid repelling boundaries. The walls of the recess 115 substantially prevent lateral movement of the first substrate 109 with respect to the holder 101, the second substrate 109 and the spacers 113. Spring-loaded compressor elements 127 are provided on the lid 102 of the holder 101 at positions corresponding to the spacers 113, to urge the first substrate 117 towards the second substrate 109.

When the holder 101 is closed, a portion of the first substrate 117 is exposed below the second substrate 109, through the window 105, to allow for sample loading. Sample liquid spreading can be observed through the window 105, and in some embodiments analyte binding may be detected through the window, without dismantling the assay assembly.

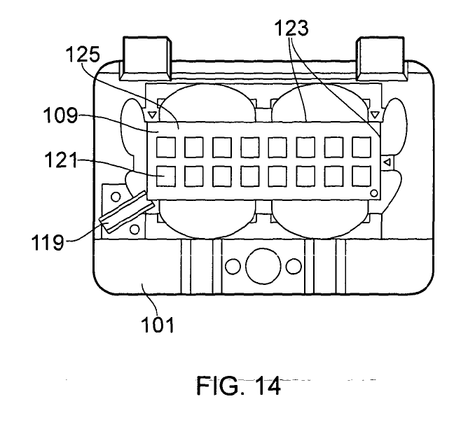

FIG. 14 shows a plan view of a portion of an assay assembly according to an embodiment of the present invention. The second substrate 109, including a plurality of microarrays 121 provided on a surface 125 thereof, is held in a recess of the holder 101. The second substrate 109 is urged towards the side walls 123 of the recess by a biasing element 119. In this way, the second substrate 109 is held snugly against the side walls 123 of the recess, substantially preventing lateral movement of the substrate with respect to the holder 101.

### EXAMPLES

### Example 1—Sample Loading

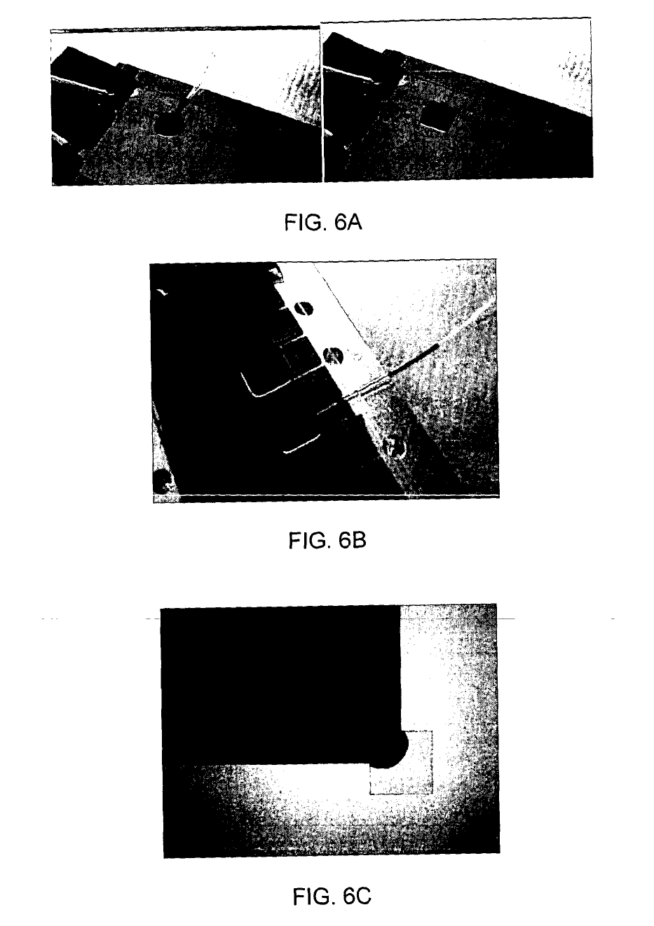

As many array sample liquids, include surfactant, providing a sample solution containing surfactant was investigated. Satisfactory sample spreading was observed for 8 μl of dye solution in PBS with 0.05% Tween, with side loading from a pipette into a hydrophilic channel, as shown in FIG. 6A. The second surface was a Menzel-Glaser slide (no array), with a 200 μm gap between the first and second surfaces. The hydrophobic boundaries were formed from CYTOP. FIG. 6B illustrates sample loading where a groove is provided. Satisfactory sample spreading is shown in FIG. 6B. The liquid is water soluble dye in PBS buffer. The hydrophobic boundaries were formed from parylene.

FIG. 6C shows improved filling to the corners of the hydrophilic surface when an overflow region is provided.

### Example 2—Sample Diffusion

In order to assess the diffusion of analytes in a liquid sample held between the first and second surfaces, a sample solution comprising fluorescently labelled BSA-AlexaFluor647 (1:1000 solution in PBS of 2 mg/ml stock of labelled protein) was loaded onto an example assembly. The non-specifically protein adsorbant surface (second surface; no array) used in these tests was home-made nitrocellulose spincoated onto silanated glass.

The surface was allowed to bind the protein for 30 min at room temperature, after which the second surface was rinsed by dipping quickly in abundant amount of PBS for 5 minutes with gentle shaking, dried and imaged on a fluorescent microarray scanner.

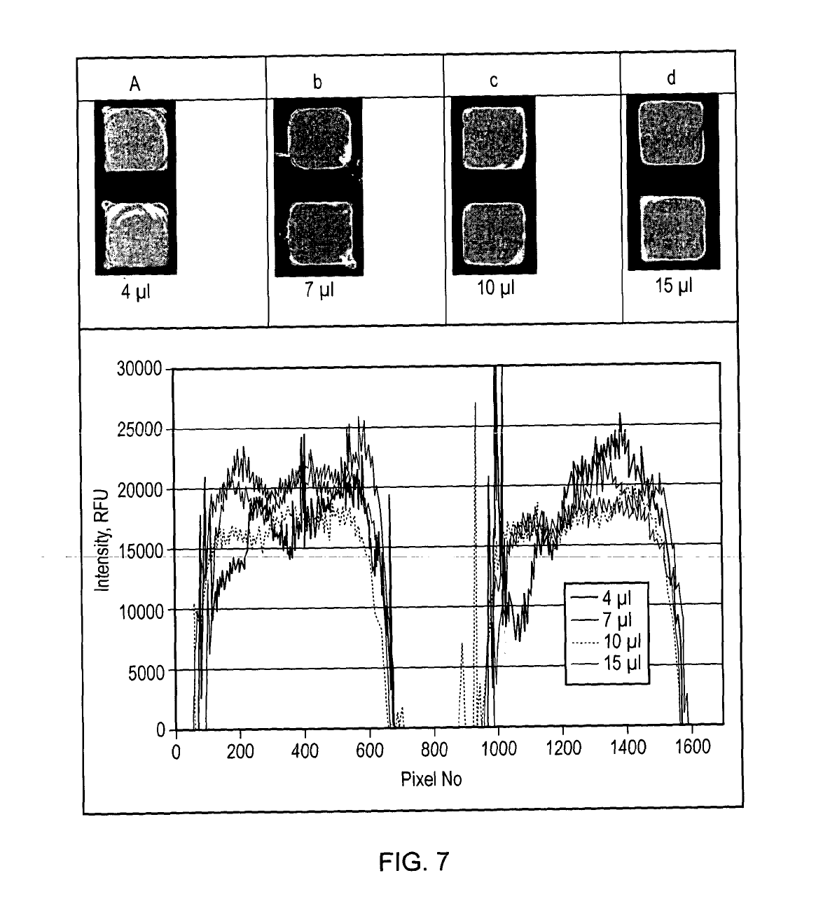

The data demonstrate that not only physical filling of the virtual ‘wells’ of the desired shape is possible, but also that fluorescently labelled BSA diffuses towards the ‘array’ surface throughout our quasi-square shape hydrophilic region in a reasonably uniform fashion. This is shown in FIG. 7, which shows that labelled BSA adsorbed to the second surface across the hydrophilic regions. Different volumes of sample liquid were supplied (FIGS. 7A-D). FIG. 7F shows intensity profiles across example “wells”, and demonstrate even sample absorption across the “well”. For smaller sample volumes, more adsorption is observed at the centre of the “well”.

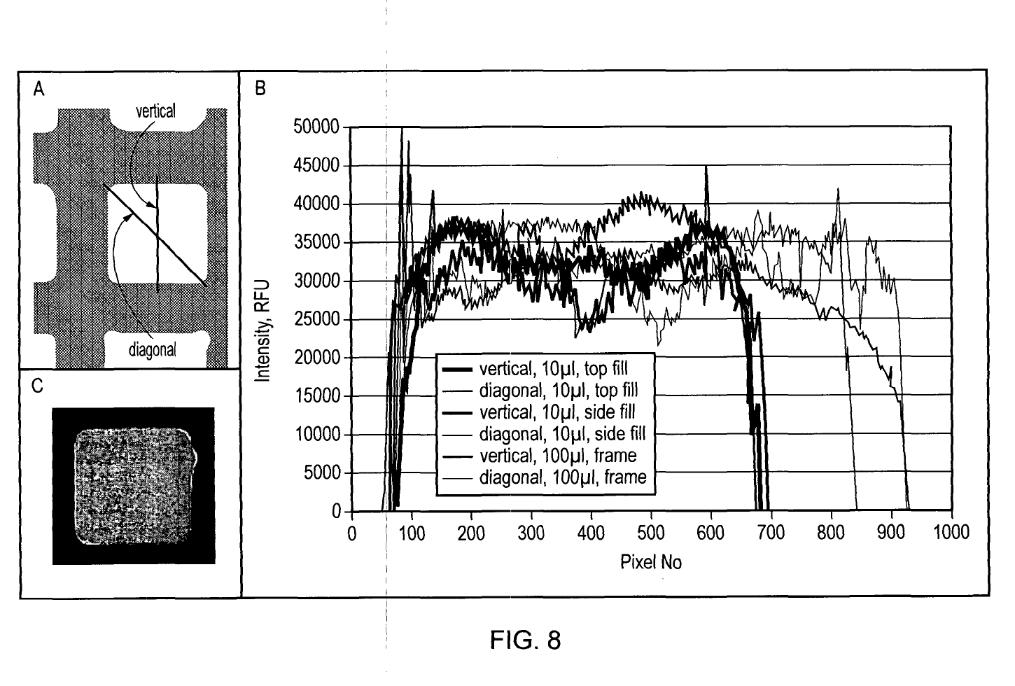

The role of the cell filling method was tested for a 10 μl cell with a piece of very fine tubing made by extruding a gel filling tip while heated, forming a thin slightly curved tip. After that the tip was used to fill the cell from the side. Filling of the cell from the side gave a slightly more even pattern of intensity than in case of ‘top-loading’, as shown in FIG. 8. This figure illustrates data from as experiment where patterns of solute diffusion for 3 different conditions of ‘well’ filling were compared: 1) sample was applied to a 10 μl cell by pre-setting the screws to give the required gap and by pipetting from the top and then lowering the array slide; 2) 10 μl of sample were applied by loading from the side into a pre-set gap on the cell with the use of a fine tip and 3) 100 μl of sample was applied in a SIMplex frame from GenTel.

The same solution of fluorescently labelled BSA was applied to home-made nitrocellulose slides from the same batch to explore the 3 loading techniques. Comparison of the patterns of intra-well intensity distribution seen in 10 μl ‘wells’ produced with an example assembly of the present invention with those observed in a conventional frame with a larger probing volume suggests that the performance of the present assembly is comparable, despite requiring much lower sample volume.

### Example 3—BSA Array Assay

A reverse-phase microarray assay, where BSA as a binding agent interacts with the probe rabbit anti-BSA serum, was chosen as a model to demonstrate the efficacy of the invention, where a planar microarray is used. Two types of arrayed patterns of BSA were used: a 10×10 array created as 16 pads spaced at 9 mm and a pattern covering nearly the whole slide solidly, generated by 4 arrayer tips, both with spot-to-spot spacing of 500 μm. BSA was spotted in PBS at 1 nl volume on Perkin Elmer Piezorray™. The blocker was β-lactoglobulin (BLG). The arrays were probed in the first reaction with rabbit anti-BSA serum followed by a wash. In the second reaction, binding was detected with anti-rabbit AlexaFluor647.

The reactions were then incubated for the desired length of time, which was one hour for the anti-BSA in the first reaction and 45 minutes for fluorescently labelled antibodies in the second reaction. Each of the 2 steps of binding reactions were done inside a humidity control box (μBox from Quantifoil Instruments GmbH was used). The use of the humidity box prevented evaporation in this case, but it is not essential. It can be helpful to reduce drying, contamination and light.

A simple calculation indicates that for a gap of 200 μm with a square hydrophilic region having a side length of 6 mm, the surface area of the liquid gas (LG) interface (SLG=4 sides×0.2 mm×6 mm) will constitute about 6% of the total surface area of the whole volume of the sample comprising both the liquid gas and the solid-liquid (SL) interface (Stotal=SLG+SSL=SLG+2 sides×6 mm×6 mm). It is possible to appreciate that the narrower the gap, the smaller the fraction of the surface from which evaporation can occur.

After the reaction was finished the upper (array) slide was lifted at horizontal orientation with the sample droplets still attached to the surface of the array. It is important to note that if the slide remains horizontal there is no cross contamination between the virtual ‘wells’ at this stage, as sample droplets would not be compelled to intermingle by any forces present. While remaining in the upside down orientation the slide was very quickly exposed to a wash solution. The initial wash and/or rinse can be carried out, for example, by squirting wash solution from the nozzle of a wash bottle upwards at the array slide so that the bulk of the sample/probe solution is removed towards the slide edge very quickly. After that initial rinse the slide was quickly immersed in a slide chamber (volume˜30 ml), or a tank (Wheeton dish, −250 ml) containing wash solution e.g. PBS or PBST (PBS with Tween added at 0.1%) and allowed to soak according to the required protocol for several minutes, e. g. 15 minutes, with shaking.

The solution was then decanted, and replaced with fresh wash solution and the slide was soaked in it again for another 15 minutes. The wash procedure was repeated one more time.

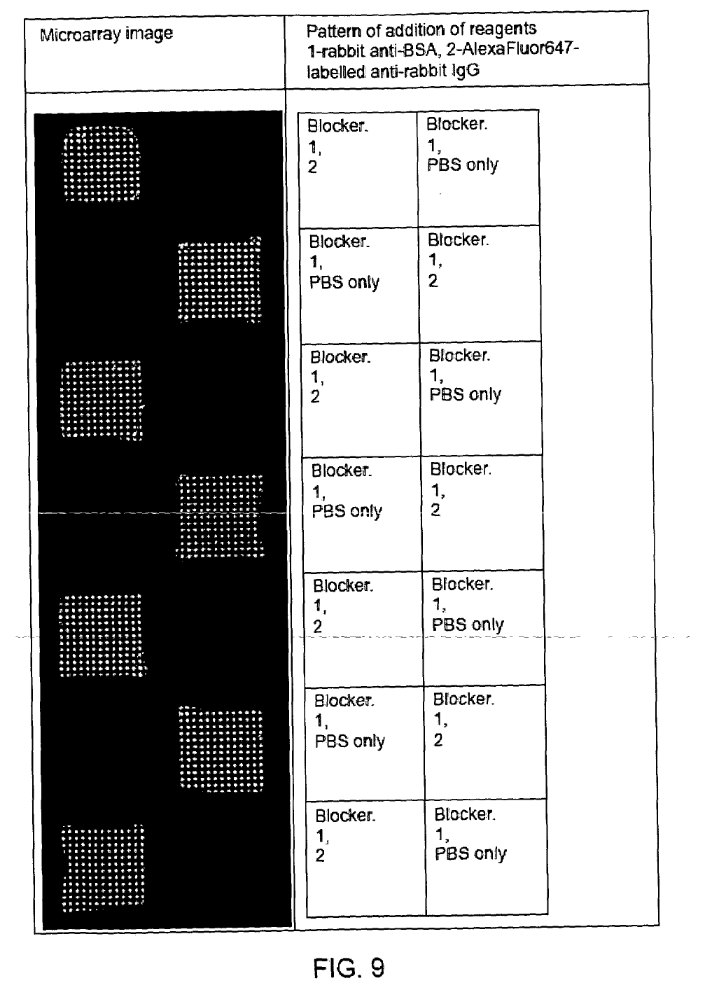

FIG. 9 shows this results of this example, and illustrates that cross-contamination was avoided. This is demonstrated on an array covering the whole surface of a slide, to which 3 different reagents, including blocker, are applied with the use of the patterned chip. For the final step the fluorescent labelling reagent is applied only to every other well. The fluorescence image of the probed slide shows there are no ‘leaks’ into neighbouring areas and/or wells filled with buffer only. In this Figure, 1 is rabbit anti-BAS, 2 is anti-rabbit AlexaFluor647, and the blocker is β-lactoglobulin (BLG).

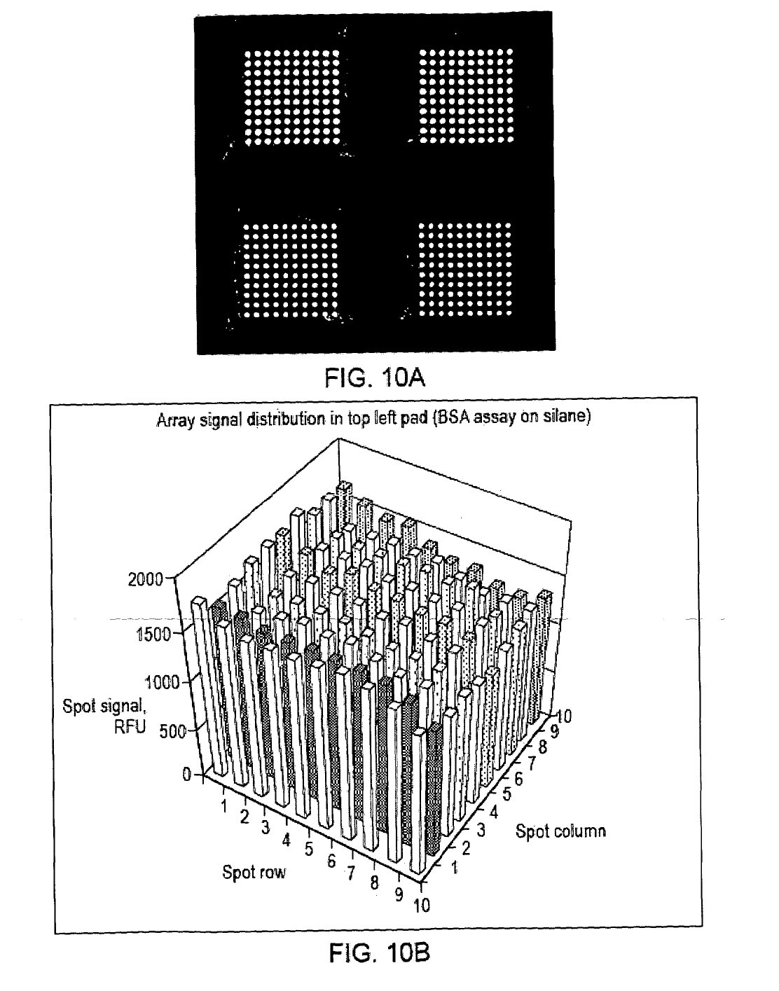

For the central portions of the array ‘pads’ created on a PATH™ slide, covering the central nearly square area with side length of 4-5 mm, good intra-well uniformity of array probing pattern could be achieved for volumes as low as 7 μl. The results are shown in FIG. 10, where FIG. 10A shows an image of the array, and FIG. 10B shows array signal distribution. The values are shown in Table 1 below:

TABLE 1

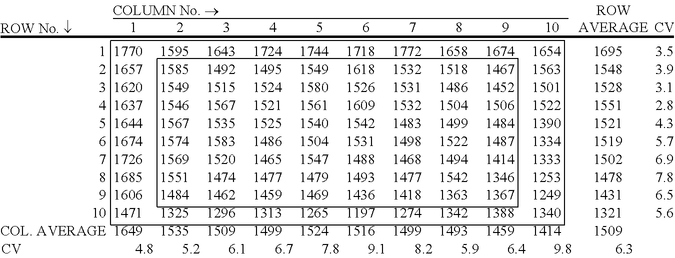

Typically outside the area covered by about 9-10 rows of spots positioned with 500 μm spacing, spot signal is lower in the rows and columns (i.e. for spots positioned at the perimeter). Without wishing to be bound by theory, one possible explanation of these edge effects is that the cell ‘perimeter’ being a liquid-gas interface is subject to the surface tension of the meniscus, which affects the diffusion of the analyte in the proximity of the meniscus.

The demonstrations of the principle of the invention with this model biological assay were achieved on the following surfaces: APTES silane (made in own lab by dip-coating), nitrocellulose (optically clear, made in own lab by spin-coating), PATH (thin commercial nitrocellulose covering the slide solidly from GenTel), and FAST (commercial thick porous nitrocellulose from Whatman, 16-pad design). The smallest ‘well’ volume of sample applied in the proof-of-principle studies for the model BSA microarray assay was 7 μl. This is about 10 times smaller volume than the minimal volume required to be used on the conventional frames to achieve similar result.

(Key steps in the array probing protocol are the same or similar to probing protocols followed with the use of conventional frames, but two adaptations need to be highlighted. Preferably, when more than one binding reaction is carried out, the last application of wash preceding application of the next sample should not contain any detergent. This can easily be achieved, for example, by performing the last wash in PBS only, After this final rinse, preferably the bulk of the wash solution clinging to the slide is removed, which is easily achieved, for example, by blowing nitrogen or dry air over the array slide for a few seconds, or by spinning the slide. This latter step should preferably be done quickly that it does not introduce any excessive drying of the slide. After these two steps the array slide is ready to be loaded onto the patterned chip filled with the next sample for the next reaction. At every stage where sample loading is involved it is possible for the operator to assess visually whether the ‘wells’ are physically separated during the course of the binding reaction.)

### Example 4—Comparison with Array Probing Frame

To exemplify the capability of an assembly embodying the present invention, a test was also carried out of a realistic and challenging (protein-protein interactions are not always strong, minimal amount of capture protein used) microarray assay. The assay involved was ERK2-His interacting with KSR-GST detected by mouse anti-GST and anti-mouse AlexaFluor647-labelled antibody. The fully processed slides were fluorescently scanned using a ScanArray Express scanner and imager to reveal the fluorescent spots which had positively interacted.

As summarised below, three ‘probing frames’: two of an example first surface of the present invention, and one commercial frame, were tested in parallel. The microarray was created as 16 sub-arrays each on a 12×12 grid (12 spots in each row) on PATH slides, each structured to the following layout:

Array Layout

| Row | | |
|---|---|---|
| No. | Spot composition | Role in array analysis |
| 1 | Anti-mouse AlexaFluor647 Ab | Positive detection and |
| | | positioning control |
| 2 | PBS spotting buffer only | Negative (buffer) control |
| 3 | BSA in PBS | Negative (interactor) control |
| 4 | ERK2-His at 0.125 mg/ml | Interactor protein |
| 5 | ERK2-His at 0.125 mg/ml | Interactor protein |
| 6 | ERK2-His at 0.125 mg/ml | Interactor protein |
| 7 | ERK2-His at 0.063 mg/ml | Interactor protein |
| 8 | ERK2-His at 0.063 mg/ml | Interactor protein |
| 9 | ERK2-His at 0.063 mg/ml | Interactor protein |
| 10 | ERK2-His at 0.125 mg/ml | Interactor protein |
| 11 | ERK2-His at 0.125 mg/ml | Interactor protein |
| 12 | ERK2-His at 0.125 mg/ml | Interactor protein |

The experiment was designed to draw a comparison between our new design (small volumes) and the conventional clip-on frame (well volume used was 70 μl). Three array slide of the same manufacturing batch were used, and subsequent reactions and washes were performed in an entirely identical manner in each case. Wells filled with the positive interactor were alternated with the wells containing ‘0 probe’ control (probing buffer 3% BSA in PBS with no protein added) to examine the possibility of cross-contamination between wells. Probe protein concentration added was the same in all wells (‘ambient analyte regime’), while the volumes added were as a particular technique required.

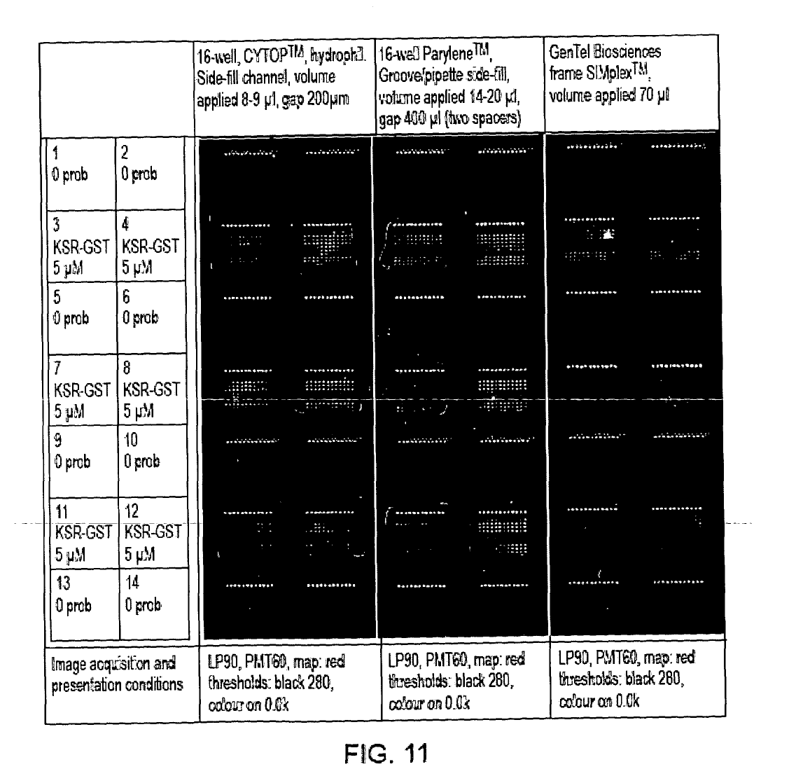

The results are shown in FIG. 11. The array images were subjected to normal spot signal quantitation protocol (built-in Quantitate function of ScanArrayExpress software). The data presented are Spot Signal-Background (as normal, representing the actual interactor spots). Details of analysis were: 300 μm spot diameter, adaptive circle mode, spot grid spacing 450 μm, 16 subarrays of 12×12 spots on a 2 col.×8 row sub-grid.

The data corresponding to rows or columns clipped by the ‘frames’ and extreme singular outliers were excluded from the statistical analysis (this is a normal approach in dealing with real-life arrays). The areas of increased background were retained in all cases to give a representation of ‘noise’ statistics for the run.

It should be noted that there is some non-process variation in the size of the printed spots, i.e. the arrays were created on a 4-dipenser tip arrayer (Piezorray™) with some inter-tip variation and also the slides may possess some slight asymmetry around the vertical axis.

The data are shown in Tables 2-7 below. Two key parameters are shown: average spot signal and the associated CV (Coefficient of Variation). Where there is interaction with the array, the higher the spot signal the better the performance, and the lower the CV the better the performance. For the negative control arrays, it is important that their signal is as low as possible.

The data indicate (Tables 2 to 7 below) that the quality of microarray probing that can be achieved on example assemblies of the present invention with small volumes is similar to the conventional frame. However, the conventional frame requires considerably larger probing volumes.

TABLE 2
ERK 0.125 mg/ml

| | | AVER. SPOT SIGNAL | | | CV (n = 33-36) | | |
|---|---|---|---|---|---|---|---|
| | PAD | ERK (1) | ERK (1) | ERK (1) | ERK (1) | ERK (1) | ERK (1) |
| PROBE | NO. | CYTOP | PARY | FRAME | CYTOP | PARY | FRAME |
| 0 probe | 1 | 44 | 37 | 29 | 19 | 26 | 20 |
| 0 probe | 2 | 57 | 45 | 29 | 22 | 31 | 23 |
| KSR-GST | 3 | 223 | 342 | 366 | 15 | 9 | 56 |
| KSR-GST | 4 | 624 | 667 | 233 | 54 | 47 | 20 |
| 0 probe | 5 | 43 | 32 | 35 | 28 | 30 | 32 |
| 0 probe | 6 | 45 | 32 | 27 | 24 | 20 | 33 |
| KSR-GST | 7 | 424 | 341 | 290 | 39 | 9 | 17 |
| KSR-GST | 8 | 696 | 823 | 319 | 27 | 16 | 20 |
| 0 probe | 9 | 45 | 35 | 32 | 34 | 20 | 24 |
| 0 probe | 10 | 38 | 38 | 32 | 16 | 16 | 24 |
| KSR-GST | 11 | 229 | 301 | 308 | 45 | 18 | 18 |
| KSR-GST | 12 | 285 | 859 | 327 | 23 | 23 | 18 |
| 0 probe | 13 | 46 | 38 | 23 | 46 | 28 | 39 |
| 0 probe | 14 | 39 | 45 | 32 | 26 | 20 | 20 |

TABLE 3
ERK 0.063 mg/ml

| | | AVER. SPOT SIGNAL | | | CV (n = 33-36) | | |
|---|---|---|---|---|---|---|---|
| | PAD | ERK(2) | ERK(2) | ERK(2) | ERK(2) | ERK(2) | ERK(2) |
| PROBE | NO. | CYTOP | PARY | FRAME | CYTOP | PARY | FRAME |
| 0 probe | 3 | 23 | 24 | 57 | 28 | 27 | 57 |
| 0 probe | 4 | 34 | 30 | 21 | 20 | 33 | 37 |
| KSR-GST | 5 | 149 | 146 | 237 | 15 | 17 | 21 |
| KSR-GST | 6 | 286 | 257 | 164 | 56 | 40 | 41 |
| 0 probe | 7 | 26 | 42 | 9 | 33 | 279 | 119 |
| 0 probe | 8 | 30 | 19 | 8 | 36 | 41 | 101 |
| KSR-GST | 9 | 173 | 152 | 132 | 35 | 27 | 16 |
| KSR-GST | 10 | 220 | 243 | 158 | 19 | 17 | 16 |
| 0 probe | 11 | 23 | 23 | 19 | 24 | 28 | 35 |
| 0 probe | 12 | 26 | 24 | 24 | 46 | 27 | 29 |
| KSR-GST | 13 | 95 | 129 | 117 | 19 | 17 | 31 |
| KSR-GST | 14 | 110 | 181 | 147 | 30 | 31 | 23 |
| 0 probe | 15 | 22 | 22 | 72 | 37 | 57 | 508 |
| 0 probe | 16 | 14 | 17 | 10 | 49 | 46 | 67 |

TABLE 4
ERK 0.125 mg/ml

| | | AVER. SPOT SIGNAL | | | CV (n = 33-36) | | |
|---|---|---|---|---|---|---|---|
| | PAD | ERK (3) | ERK (3) | ERK (3) | ERK (3) | ERK (3) | ERK (3) |
| PROBE | NO. | CYTOP | PARY | FRAME | CYTOP | PARY | FRAME |
| 0 probe | 1 | 38 | 39 | 29 | 19 | 21 | 33 |
| 0 probe | 2 | 58 | 45 | 30 | 16 | 23 | 33 |
| KSR-GST | 3 | 227 | 356 | 462 | 16 | 16 | 16 |
| KSR-GST | 4 | 359 | 552 | 357 | 20 | 15 | 32 |
| 0 probe | 5 | 36 | 29 | 31 | 36 | 46 | 26 |
| 0 probe | 6 | 39 | 23 | 26 | 27 | 32 | 29 |
| KSR-GST | 7 | 304 | 382 | 291 | 18 | 22 | 17 |
| KSR-GST | 8 | 447 | 472 | 414 | 14 | 6 | 56 |
| 0 probe | 9 | 40 | 31 | 34 | 19 | 27 | 51 |
| 0 probe | 10 | 37 | 34 | 50 | 22 | 19 | 28 |
| KSR-GST | 11 | 235 | 285 | 265 | 12 | 11 | 22 |
| KSR-GST | 12 | 291 | 444 | 329 | 37 | 47 | 20 |
| 0 probe | 13 | 51 | 48 | 53 | 27 | 32 | 173 |
| 0 probe | 14 | 45 | 47 | 40 | 36 | 22 | 28 |

TABLE 5
PBS CONTROL

| | | AVER. SPOT SIGNAL | | | CV (n = 11-12) | | |
|---|---|---|---|---|---|---|---|
| | PAD | PBS | PBS | PBS | PBS | PBS | PBS |
| PROBE | NO. | CYTOP | PARY | FRAME | CYTOP | PARY | FRAME |
| 0 probe | 1 | −1 | 22 | 2 | −1537 | 267 | 520 |
| 0 probe | 2 | 8 | 5 | 43 | 131 | 147 | 165 |
| KSR-GST | 3 | 14 | 29 | 31 | 135 | 72 | 118 |
| KSR-GST | 4 | 39 | 45 | 22 | 124 | 52 | 242 |
| 0 probe | 5 | −1 | 3 | 4 | −834 | 226 | 152 |
| 0 probe | 6 | 6 | 5 | 5 | 168 | 92 | 527 |
| KSR-GST | 7 | 19 | 32 | 10 | 105 | 41 | 108 |
| KSR-GST | 8 | 37 | 47 | 15 | 42 | 66 | 49 |
| 0 probe | 9 | 6 | 7 | 2 | 104 | 103 | 287 |
| 0 probe | 10 | 3 | 8 | 352 | 219 | 73 | 352 |
| KSR-GST | 11 | 19 | 29 | 18 | 83 | 59 | 46 |
| KSR-GST | 12 | 19 | 46 | 21 | 94 | 44 | 74 |
| 0 probe | 13 | 2 | 1 | 611 | 302 | 1438 | 611 |
| 0 probe | 14 | 2 | 6 | 3 | 369 | 134 | 316 |

TABLE 6
BSA CONTROL

| | | AVER. SPOT SIGNAL | | | CV (n = 11-12) | | |
|---|---|---|---|---|---|---|---|
| | PAD | BSA | BSA | BSA | BSA | BSA | BSA |
| PROBE | NO. | CYTOP | PARY | FRAME | CYTOP | PARY | FRAME |
| 0 probe | 1 | 36 | 13 | 21 | 78 | 58 | 91 |
| 0 probe | 2 | 32 | 26 | 18 | 28 | 45 | 40 |
| KSR-GST | 3 | −10 | 89 | 14 | −180 | 18 | 191 |
| KSR-GST | 4 | 98 | 149 | 8 | 89 | 18 | 298 |
| 0 probe | 5 | 26 | 24 | 22 | 23 | 26 | 28 |
| 0 probe | 6 | 29 | 29 | 25 | 48 | 29 | 31 |
| KSR-GST | 7 | −34 | 97 | 20 | −76 | 19 | 95 |
| KSR-GST | 8 | 174 | 222 | 63 | 25 | 16 | 99 |
| 0 probe | 9 | 21 | 17 | 19 | 28 | 35 | 50 |
| 0 probe | 10 | 24 | 23 | 20 | 52 | 37 | 37 |
| KSR-GST | 11 | −1 | 161 | 66 | −1411 | 24 | 52 |
| KSR-GST | 12 | 23 | 246 | 121 | 194 | 50 | 68 |
| 0 probe | 13 | 25 | 125 | 19 | 39 | 288 | 26 |
| 0 probe | 14 | 27 | 33 | 28 | 43 | 37 | 38 |

TABLE 7
Ab CONTROL

| | | AVER. SPOT SIGNAL | | | CV (n = 11-12) | | |
|---|---|---|---|---|---|---|---|
| | PAD | Fluor Ab | Fluor Ab | Fluor Ab | Fluor Ab | Fluor Ab | Fluor Ab |
| PROBE | NO. | CYTOP | PARY | FRAME | CYTOP | PARY | FRAME |
| 0 probe | 1 | 15490 | 11926 | 11688 | 8 | 6 | 5 |
| 0 probe | 2 | 14130 | 12424 | 10650 | 5 | 7 | 4 |
| KSR-GST | 3 | 20090 | 17077 | 13696 | 7 | 9 | 4 |
| KSR-GST | 4 | 20851 | 16093 | 13575 | 8 | 8 | 3 |
| 0 probe | 5 | 21522 | 19139 | 17762 | 7 | 16 | 4 |
| 0 probe | 6 | 22084 | 17198 | 18045 | 9 | 11 | 8 |
| KSR-GST | 7 | 10538 | 7882 | 7383 | 9 | 10 | 17 |
| KSR-GST | 8 | 9254 | 7403 | 7098 | 14 | 11 | 15 |
| 0 probe | 9 | 7864 | 5991 | 5991 | 13 | 7 | 4 |
| 0 probe | 10 | 7209 | 6233 | 5763 | 6 | 7 | 4 |
| KSR-GST | 11 | 16509 | 12680 | 13056 | 11 | 9 | 10 |
| KSR-GST | 12 | 16717 | 12166 | 12251 | 7 | 8 | 13 |
| 0 probe | 13 | 18875 | 16611 | 14757 | 10 | 8 | 10 |
| 0 probe | 14 | 19159 | 16644 | 15851 | 7 | 4 | 9 |

### Example 5—Comparison with LifterSlip

A comparison of an assembly embodying the present invention (having a single surface wettable region), with a similar assay covered with a “LifterSlip” was carried out.

The array type was protein-protein interaction, using 48 (4×12) blocks of 10×10 spots. There were 88 capture spots of ERK2 per block. The area probed was 20 mm×60 mm. The spacer gap was 62 μm. The surface of the array slide was PATH (thin nitrocellulose). 2% BSA in PBS was supplied as a blocker, and the probe used was 10 μM KSR(ERK2 binding domain)-GST in 2% BSA in PBS. The detection step employed mouse anti-GST+anti-mouse AlexaFluor647. The sample volume applied was 75 μl in both cases.

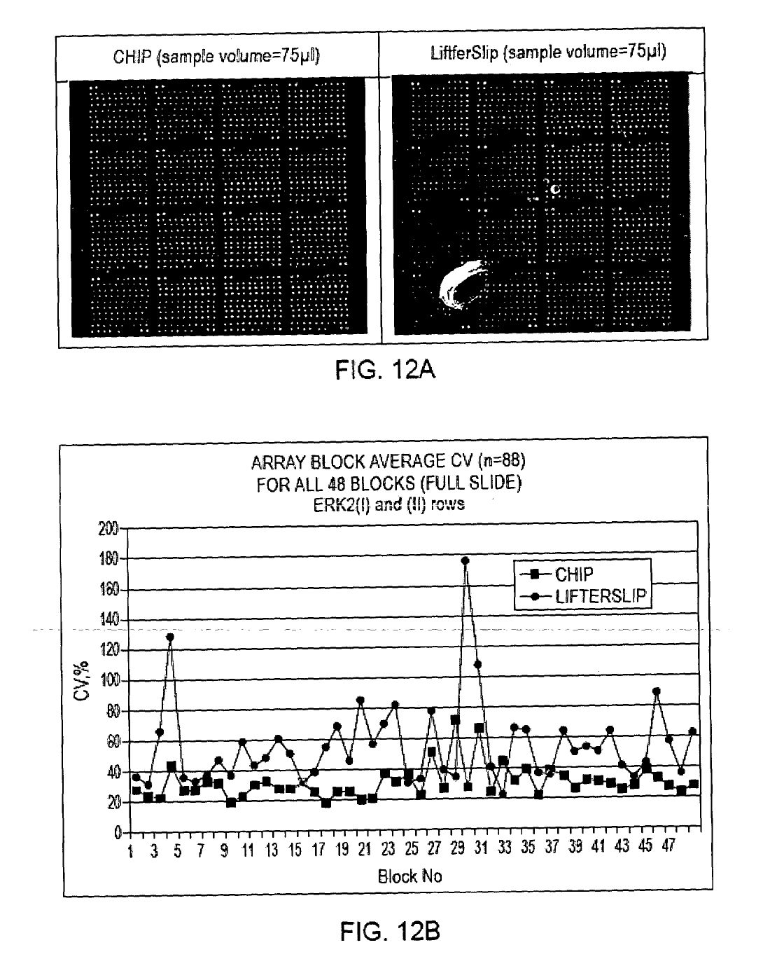

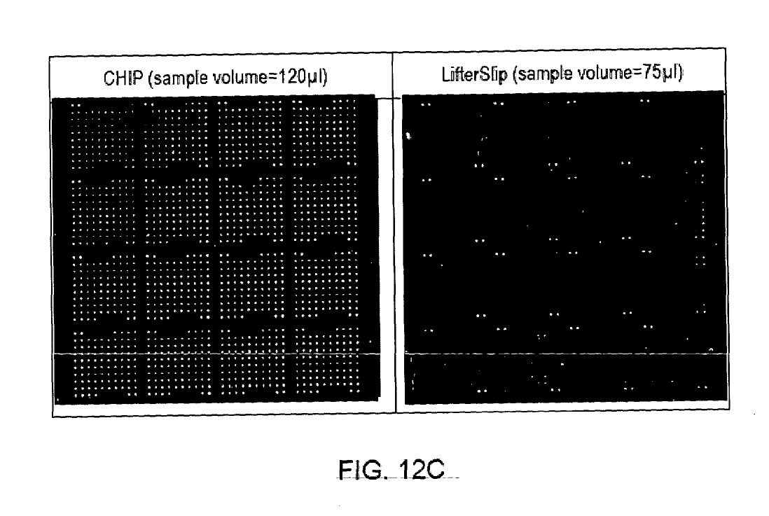

This test indicated that using a first surface according to the present invention, rather than a LifterSlip, enhances uniformity of microarray probing. The array images are shown in FIG. 12A. The central portion of the array slide is shown (array blocks 17-32), which is an area of about 20 mm×20 mm. The images show a more uniform array probing response, with fewer artefacts for the assembly of the present invention (labelled “CHIP”).

Additionally, the parameter ‘block average CV’ (each for 88 interactor spots) (CV is coefficient of variation, which is the standard deviation divided by average*100%) was charted for all 48 array blocks, and shows that array data with a first surface according to the present invention are significantly less variable, as shown in FIG. 12B. The average CV for 48 blocks was 55 for the LifterSlip, and 31 where a first surface according to the present invention was used.

This reduction of the statistical ‘noise’ leads to more reliable identification of protein-protein interactions on arrays.

Results of a similar protein-protein interaction array are shown in FIG. 12C. The arrays used were the same as those used for FIG. 12A. However, the sample volumes applied were different. The LifterSlip does not allow more sample to be applied in a controlled way, so again a sample volume of 75 μl was applied. In contrast, the gap between the first and second surfaces of an assembly embodying the present invention was adjusted, to allow a sample volume of 120 μl to be applied. FIG. 12C shows that here, the assembly embodying the present invention produced dramatically better data quality. This illustrates that varying the gap between the first and second surfaces can be advantageous.

These embodiments have been described by way of example only. Modifications of these embodiments, further embodiments and modifications thereof will be apparent to the skilled person on reading this disclosure and as such are within the scope of the present invention.

All references cited herein are hereby incorporated herein by reference in their entirety for all purposes.

### REFERENCES

1. R. Ekins, F. Chu and E. Biggart, Development of Microspot Multi-Analyte Ratiometric Immunoassay Using Dual Fluorescent-Labelled Antibodies, Analytica Chimica Acta, 227, 1989, 73-96
2. M. Schena, D. Shalon, R. W Davis, P. O. Brown, Quantitative Monitoring of Gene Expression Petterns with a Complementary DNA Microarray, Science, 270, 1995, 467-470
3. P. F. Predki, Functional protein microarrays: ripe for discovery, Curr. Op. Chem. Biol., 8, 2004, 8-13
4. M. Templin, D. Stoll, J. Bachmann and T. O. Joos, Protein Microarrays and Multiplexed sandwich Immunoassays: What beats the Beads?, Combinatorial Chemistry and High Throughput Screening, 7, 2004, 223-229
5. P. Angenendt, Progress in Protein and Antibody Microarray technology, Drug Discovery Today, 10(7), 2005, 503-511
6. P. Cullen and S. Lorkowski, DNA Microarrays in medicine: Manufacturing techniques and Potential applications, Expert Opin. Ther. Patents, 12(12), 2002, 1-12
7. K.-B. Lee, S.-J. Park, C. A. Mirkin, J. C. Smith, M. Mrksich, Protein nanoarrays Generated By Dip-Pen Nanolithography, Science, 295, 2002, 1702-1705
8. J. M. Nam, S. W. Han, K.-B. Lee, X. Liu, M.A. Ratner, C. A. Mirkin, Bioactive Protein Nanoarrays on Nickel Oxide Surfaces Formed by Dip-Pen Nanolithography, Angew. Chem. Int. Ed., 43, 2004, 1246-1249
9. S.-W. Lee, B.-K. Oh, R. G. Sanedrin, K. Salalta, T. Fujigaya, C. A. Mirkin, Biologically Active Protein Nanoarrays Generated Using Parallel Nanolithography, Adv. Mater., 18, 2006, 1133-1136
10. S. Bouaidat, O. Hansen, H, Bruus, C. Berendsen, N. K. Bau-Madsen, P. Thomsen, A. Wolff and J. Johnsmann, Surface-directed capillary system: theory, experiments and applications, Lab. Chip, 5, 2005, 827-836
11. Fei Su, Krishenedu Chakrabarty and Richard Fair, Microfluidics-based biochips; technology issues, implementation platforms, and design-automation challenges, IEEE Transactions on Computer-Aided Design of integrated Circuits and Systems, 25 (2), 2006, 211-223
12. Catherine Situma, Masahiko Hashimoto and Steven A. Soper, Merging microfluidics with microarray—based bioassays, Biomolecular Engineering, 23, 2006, 213-231
13. Uwe Müller, Evolution of High-Sensitivity Microarray Technology, JALA, 8, 2003, 96-100
14. David Thompson, Ekaterina O. McKenna, Andrew Pitt and Duncan Graham, Microscale mesoarrays created by dip-pennanolithography for screening of protein-protein interactions, Biosensors and Bioelectronics, 26, 2011, 4667-4673

1. An assay assembly comprising
a first surface having at least one sample liquid wettable surface region defined by a sample liquid repelling boundary; and
a second surface, opposed to the first surface, having analyte binding agent immobilised thereon, the immobilised binding agent being provided on the second surface in one or more two-dimensional arrays of discrete array elements, wherein at least two different array elements of the two dimensional array comprise different immobilised binding agent.

2. An assay assembly according to claim 1 wherein the second surface has two or more two dimensional arrays of discrete array elements of immobilised binding agent.

3. An assay assembly according to claim 1, wherein the first surface has a plurality of sample liquid wettable surface regions, each defined by a sample liquid repelling boundary.

4. An assay assembly according to claim 1 wherein each sample liquid wettable surface region is a hydrophilic surface region, and wherein each sample liquid repelling boundary is a hydrophobic boundary.

5. An assay assembly according to claim 1 wherein each sample liquid wettable surface region has at least two dimensions of at least 0.5 mm.

6. An assay assembly according to claim 1, wherein the first surface has one or more inlet channels, for loading sample liquid into the sample liquid wettable surface regions.

7. An assay assembly according to claim 1 wherein at least one of the sample liquid wettable surface region includes one or more overflow regions.

8. An assay assembly according to claim 1 wherein each sample liquid wettable surface region requires a sample liquid volume of 60 μl or less.

9. An assay assembly according to claim 1 wherein the second surface has at least one sample liquid wettable surface region defined by a sample liquid repelling boundary.

10. An assay assembly according to claim 1, wherein the first surface is a surface of a first substrate and the second surface is a surface of a second substrate.

11. An assay assembly according to claim 10, wherein the first and second substrates are held in a holder, comprising a first substrate receiving site which holds the first substrate and a second substrate receiving site which holds the second substrate.

12. An assay assembly according to claim 11 wherein the first and second substrate receiving sites are configured to substantially prevent lateral movement of the first and second substrate with respect to the holder.

13. (canceled)

14. A kit of parts comprising:
a first substrate having a first surface, wherein the first surface has at least one sample liquid wettable surface region defined by a sample liquid repelling boundary; and
a holder having a first substrate receiving site for holding the first substrate and a second substrate receiving site for holding a second substrate,
wherein the first and second substrate receiving sites are arranged or arrangeable to hold the first and second substrates in a position wherein a surface of the second substrate is opposed to the first surface.

15. A method of determining one or more properties of a sample liquid, the method comprising:
providing a sample liquid between a first surface and a second surface, wherein the first surface has at least one sample liquid wettable surface region defined by a sample liquid repelling boundary, and wherein the second surface is opposed to the first surface and has analyte binding agent immobilised thereon; and
determining one or more properties of the liquid sample by detecting binding of analyte to the immobilised analyte binding agent.

16. A method according to claim 15 wherein the property or properties of the sample liquid determined in the method are selected from
the presence or absence of one or more analytes in the sample liquid;
the concentration of one or more analytes in the sample liquid; and
a property of one or more analytes in the liquid.
